SEQUENCE LISTING (6 sequences)

SEQ ID NO: 1 - DNA, 20 bp, Artificial
  synthesized oligonucleotide primer
ctttgaggctgacctgaagc

SEQ ID NO: 2 - DNA, 20 bp, Artificial
  synthesized oligonucleotide primer
ctgggaagaactcagcgaac

SEQ ID NO: 3 - DNA, 21 bp, Artificial
  synthesized oligonucleotide primer
ggaggagctgctgaagatgtg

SEQ ID NO: 4 - DNA, 22 bp, Artificial
  synthesized oligonucleotide primer
tggttctcccgagaggtaaaga

SEQ ID NO: 5 - DNA, 17 bp, Artificial
  synthesized oligonucleotide probe
ccagtgaagaaagtgtc

SEQ ID NO: 6 - DNA, 16 bp, Artificial
  synthesized oligonucleotide probe
cagtgaagcaagtgtc

(19) United States
(12) Patent Application Publication
(10) Pub. No.: US 2007/0134709 A1
(43) Pub. Date: Jun. 14, 2007

(54) Usages of MTHFR gene polymorphisms in predicting homocysteine level, disease risk, and treatment effects and related methods and kit

(76) Inventors: Xiping Xu, Wilmette, IL (US); Zhian Fang, Hefei (CN); Shanqun Jiang, Hefei (CN); Binyan Wang, Hefei (CN); Jianhua Yang, Hefei (CN); Shanchun Zhang, Hefei (CN); Guangyun Mao, Hefei (CN); Houxun Xing, Beijing (CN); Ping Liu, Beijing (CN); Yan Wang, Beijing (CN); Tonghua Zang, Hefei (CN); Mengde Wang, Beijing (CN); Yu Wang, Beijing (CN); Chengxiang Dai, Beijing (CN); Kerong Zhang, Beijing (CN)

Correspondence Address: Xiping Xu, 2611 Laurel Lane, Wilmette, IL, 60091, US

(21) Appl. No.: 11/638,634

(22) Filed: Dec. 13, 2006

(30) Foreign Application Priority Data
Mar. 17, 2006 (CN) 200610064915.4
Jun. 27, 2006 (CN) 200610090093.7
Dec. 14, 2005 (CN) 200510130528.1

(51) Int. Cl. C12Q 1/68; C12P 19/34; C07H 21/04; A61K 31/401; C12M 3/00

(52) U.S. Cl. 435/6; 536/24.3; 435/91.2; 435/287.2; 977/924; 514/423

(57) ABSTRACT

This invention features our discovery on usages of Methylenetetrahydrofolate Reductase (MTHFR) gene polymorphisms in predicting homocysteine (Hcy) level and/or incidence and prognosis of diseases associated with increased Hcy level in a subject, as well as predicting treatment effects of medicines in the category of Angiotension Converting Enzyme Inhibihor (ACEI) with and without combination with B Vitamins. This invention also features our discovery on laboratory and analytical methods that are essential to the above described usages of MTHFR gene polymorphisms. In addition, this invention features a kit that has translated the above discoveries into a practical and reliable tool that can be applied to accomplish the above described usages of MTHFR gene polymorphisms. This invention represents an important step in realizing personalized medicine, with the goal to tailor diagnosis, prevention and treatment strategy to meet individual needs.

### OTHER REFERENCES

Not Applicable.

### STATEMENT REGARDING FEDERALLY SPONSORED RESEARCH OR DEVELOPMENT

Not Applicable.

### REFERENCE TO SEQUENCE LISTING

Statement: the “Sequence Listing” on the paper and on the computer readable 3.5 inch diskette are the same.

### FIELD OF THE INVENTION

This invention features our discovery on usages of Methylenetetrahydrofolate Reductase (MTHFR) gene polymorphisms in predicting homocysteine (Hcy) level and/or incidence and prognosis of diseases associated with increased Hcy level in a subject, as well as in predicting treatment effects of medicines in the category of Angiotension Converting Enzyme Inhibihor (ACEI) with and without combination with B Vitamins. The invention also features our discovery on laboratory and analytical methods that are central to the above described usages of MTHFR gene polymorphisms. In addition, this invention features a kit that has translated the above discoveries into a practical and reliable tool that can be applied to accomplish the above described usages of MTHFR gene polymorphisms. This invention represents an important step in realizing personalized medicine, with the goal to tailor diagnosis, prevention and treatment strategy to meet individual needs.

### BACKGROUND OF THE INVENTION

Homocysteine (Hcy) is a sulfur-containing amino acid formed during the metabolism of methionine. Hcy is metabolized by one of two pathways: remethylation and transsulfuration. In the remethylation cycle, around 50% of Hcy is salvaged by the acquisition of a methyl group in a reaction catalyzed by methionine synthase. Vitamin B12 is an essential cofactor for methionine synthase, N5-methyl-tetrahydrofolate is the methyl donor in this reaction, and N5, N10-methylenetetrahydrofolate reductase functions as a catalyst in the remethylation process.

Under conditions in which an excess of methionine is present or cysteine synthesis is required, the remaining Hcy enters the transsulfuration pathway. In this pathway, Hcy condenses with serine to form cystathionine in a reaction catalyzed by the vitamin B6-dependent enzyme cystathionine b-synthase. Cystathionine is subsequently hydrolyzed to form cysteine, which may in turn be incorporated into glutathione or further metabolized to sulfate and excreted in the urine.

Elevations in plasma Hcy are typically caused either by genetic defects in the enzymes involved in homocysteine metabolism or by nutritional deficiencies in B vitamin cofactors. Other factors such as disease status and medication are also involved in altering plasma Hcy level. Recently, more evidence showed that a homozygous deficiency of MTHFR, the major enzyme involved in the remethylation of homocysteine to methionine, may lead to severe hyperhomocysteinemia. Kang and colleagues reported a thermolabile variant of N5, N10-methylenetetrahydrofolate reductase caused by a point mutation (C677T) in the coding region for the N5, N10-methylenetetrahydrofolate binding site, leading to the substitution of valine for alanine. [Brattstrom et al., Circulation, 1998, 98(23):2520-2526]. A meta-analysis investigated 13 studies published during the past 3 years, in which there were measurements of plasma homocysteine in relation to the MTHFR C677T genetic variant, which comprised 5869 genotyped cardiovascular disease patients and 6644 genotyped control subjects. The analyses concluded that the C677T/MTHFR mutation is a major cause of mild hyperhomocysteinemia.

Hyperhomocysteinemia is associated with many types of vascular diseases and their complications. The potential mechanisms are that hyperhomocysteinemia may cause vascular endothelial impairment and dysfunction, stimulate proliferation of vascular smooth muscle cell, increase platelet adhesiveness, enhance LDL oxidation and deposition in the arterial wall, and direct activation of the coagulation cascade. Studies identified MTHFR gene as a susceptibility gene of pregnancy induced hypertensive disease [Keshen Li et al.,Chinese Journal of Public Health, 2004, 20, 6 : 762-764]. To date, elevated homocysteine concentration has been generally considered to be a significant, independent risk factor for atherothrombotic vascular disease. An increase in circulating homocysteine level may be linearly associated with risk of coronary heart disease, cerebrovascular disease, and occlusive vascular disease.

Sutton's study, [Sutton et al., Circulation, 1997, 96: 1745] suggested that elevated levels of homocysteine may be related to the cause of isolated systolic hypertension (ISH). Subsequent studies reported that MTHFR gene is a susceptibility gene of ISH. Hyperhomocysteinemia might be an important intermediate phenotype [Xiaonan Sun et al., 2003, 31(4): 269-273]. [Lim et al., Am J Epidemiol, 2002, 156: 1105-1113]. Hyperhomocysteinemia was also associated with an increased risk of hypertension. Moreover, it's reported [Cesari M er al., Arterioscler Thromb Vasc Biol 2005, 25:115-121] that Hyperhomocysteinemia was the strongest predictor (P=0.001) of a low (<40%) left ventricular ejection fraction (LVEF). Elevated Hcy level in patients with hypertension might be a crucial, independent predictor for target organ damage, such as heart, brain, and kidney.

Jacques and colleagues [Jacques PF et al., Am J Clin Nutr. 1999, 69(3):482-489], using surplus sera from phase 2 of the third National Health and Nutrition Examination Survey, measured serum total homocysteine concentrations for a nationally representative sample of 3766 males and 4819 females aged ≧12 y. They found that age-adjusted geometric mean total homocysteine concentrations were 9.6 and 7.9 mmol/L in non-Hispanic white males and females, 9.8 and 8.2 mmol/L in non-Hispanic black males and females, and 9.4 and 7.4 mmol/L in Mexican American males and females, respectively. Wang et al investigated 1168 subjects, including both males and females aged 35-64, in population of urban and rural areas in Beijing, China. They found that geometric mean of serum homocycteine was 15.4 micromol/L in males and 12.2 micromol/L in females (P<0.001). There was a significant difference in homocysteine levels between urban population and rural population. Men from rural area had 1.5 times higher homocyteine than from urban (18.0 micromol/L vs 12.0 micromol/L, P<0.001), while the rural women had 1.3 times higher homocysteine level than urban women. The prevalence rate of hyperhomocysteinemia that defined as Hcy=16 mol/L was 15.3% in population of Beijing area. Multivariate analysis showed that gender, residential location (urban or rural), smoking and education had independent effects on level of serum homocysteine. They conclude that population in Beijing had higher serum level of homocysteine than some western countries. Gender, geographic distribution, smoking and education had some influences on homocysteine level.

In the clinical setting, stroke has increasingly become a severe life-threatening disease, characterized by high incidence, high recurrence rate, high mutilation, and high fatality rate. So far many known disease-related risk factors can be categorized into controllable and uncontrollable factors. Controllable risk factors are the main focus of primary prevention, especially involving diet modification, and drug intervention for hypertension, hyperlipidemia, and hyperglycemia, smoking and drinking abatement, and therapy for atrial fibrillation and carotid artery stenosis. However, there are plenty of clinical cases, in which subjects did not have identifiable risk factors before the occurrence of artherosclerosis-caused strokes, such as history of hypertension, diabetes mellitus and heart disease, and other habit disturbances. It underscores that more sensitive and early detection method is needed.

Most of meta-analyses revealed that hyperhomocysteinemia is an important, independent risk factor for cardiovascular disease. Patient with hyperhomocysteinemia tend to have early onset coronary artery atherosclerotic lesion, and ischemic cerebrovascular disease. Boushey et al [Boushey et al JAMA, 1995, 274(8): 1049-1057] summarized 27 published studies and found that an increase of Hcy level is a crucial predictor of the risk for fatal and nonfatal atherosclerotic vascular disease. Hypercholesteremia, as an emerging risk factor, along with other conventional factors such as hypertension and smoking, is of importance for contributing to cardiovascular diseases. A dose-dependent linear relation of Hcy level with risk of vascular disease was also found. A European Concerted Action Project on “elevated Hcy level and vascular disease” conducted simultaneously in 19 centers, and identified that Hcy is an independent risk factor for ischemic cardiovascular disease.

In plasma there exist three forms of Hcy. Homocysteine in the reduced (sulfhydryl) form, homocysteine in the oxidized (disulfide) form, its homologs: cysteine and cystine. In subjects with normal Hcy level, about 70-80% of the total Hcy is bound to protein by a disulfide linkage. With elevated Hcy levels, the percentage of Hcy in the sulfhydryl form can increase to 10-25% of the total Hcy. The total Hcy is measured as the free thiol, which is obtained by reduction. Methods used to assay Hcy include gas chromatography with mass spectroscopy, HPLC with or without fluorescence detection, and HPLC with electrochemical detection, immunological analyses, including an enzyme-linked immunoassay and an automated fluorescence polarization analyzer. Presently in clinical setting, HPLC and GC-MS methods are widely used. However, its shortcoming was also obvious, including multi-steps operation, time consuming, unreliable reproducibility, and higher cost.

Homocysteine is also closely associated with cardiovascular disease development and prognosis. In present, due to the limitation of measurement method for homocysteine level, it's necessary to setup a practical and reliable approach to predict homocysteine level, in order to early detect, prevent, and intervene with hyperhomocysteinemia and associated diseases. This invention has provided necessary methods and kit for predicting homocysteine level, and for predicting homocysteine-associated cerebrocardiovascular diseases.

Cerebrovascular disease, in particularly, stroke, is characterized by 4 major features: high incidence, high recurrence rate, high mutilation rate, and high lethality rate. Stroke is considered as one of severe life-threatening diseases. It is important to accurately predict risk factors of stroke. Known conventional risk factors include hypertension, hyperlipidemia and hyperglycaemia. However, these risk factors can not identify all the subjects at risk for stroke and constitute relatively late stage conditions. In order to early detect and prevent stroke, and improve prognosis of cerebrocardiovascular disease, it is necessary to establish a practical and reliable method for predicting important risk factors such as Hcy level.

Hypertension is a common chronic disease all over the world. Its incident is as high as 31.3% in China. Hypertension is one of major causes for life-threatening cardio- and cerebrovascular diseases. Blood pressure level is linearly correlated with prevalence of cerebro and cardiovascular diseases. Epidemiology studies showed that an increase of blood pressure level is an independent risk factor for stroke and coronary artery disease. Hence, effective control of blood pressure level is of great significance to prevent the development and improve prognosis of cerebro and cardiovascular diseases and associated complications. There are 6 types of antihypertensive drugs widely used for hypertension treatment: diuretic, beta-adrenoceptor blocker, alpha-adrenoceptor blocker, calcium antagonist, angiotensin converting enzyme inhibitor (ACEI), and angiotensin II receptor antagonist. However, in clinical practice, the therapeutic efficacy of anti-hypertensive drugs is not totally satisfactory. The reasons for inadequate therapeutic efficacy include drug intolerance, poor compliance, and mono-drug therapy (only 40%-50% control rate). More importantly, inter-individual genetic variation contributes to both therapeutic efficacy, tolerance, and adverse responses. Family-based study and twin study identified that around 30%˜60% of blood pressure variation may be attributed to genetic effect [Harrap SB, Lancet, 1994; 344: 169-171]. Drug response related genetic polymorphisms generally involve drug-metabolizing enzyme polymorphism and drug receptor polymorphism. These polymorphic loci located in drug relevant genes might be main cause of differential efficacy and safety in individuals [Sander C, Science, 2000; 287: 1977-1978][Inazu A, J Clin Invest, 1994; 94: 1872-1882].

Single nucleotide polymorphism (SNP) is DNA sequence variations that occur when a single nucleotide (A, T, C, or G) in genome sequence is altered. It is estimated that the frequency of SNP occurs in as many as 1 in 1000 base pairs. For any two unrelated individuals, there are around 3 millions of SNP differences between them. Major genetic factors influencing drug efficacy may be involved in pre-drug stimulation, drug-target binding ability and activity, drug metabolism, degradation and excretion, while drug safety is more dependent on drug metabolism, degradation and excretion, and non-specific combination and activity with other ligands. Any SNP occurring in above these processes can lead to differential drug response in individuals. For instance, the genetic-related difference in rate of side-effect between individuals can reach as high as 300 times.

In the present, the number of hypertension-related susceptibility genes is more than 70. Most studies concentrated on observing the relationship of gene polymorphism to antihypertensive drug-induced cardiovascular effects [Cusi D et al., Lancet, 1997; 349: 1353-7]. Heterozygous hypertensive patients showed a greater fall in mean arterial pressure in response to 2 months' treatment with hydrochlorothiazide than did 37 wild-type homozygous hypertensive patients (mean decrease 14.7 [2.2] vs 6.8 [1.4] mm Hg; p=0.002). It suggested that the a-adducin polymorphism (Gly 460 Trp) may identify hypertensive patients who will benefit from diuretic treatment [Schelleman H et al., Drugs, 2004; 64: 1801-1816]. Hypertensive patients with the 460W allele of the α-adducin gene had a lower risk of myocardial infarction and stroke when treated with diuretics compared with other antihypertensive therapies. With regard to blood pressure response, interactions were found between genetic polymorphisms for endothelial nitric oxide synthase and diuretics, the α-adducin gene and diuretics, the α-subunit of G protein and β-adrenoceptor antagonists, and the ACE gene and angiotensin II type 1 (AT1) receptor antagonists. Studies found an interaction between ACE inhibitors and the ACE insertion/deletion (I/D) polymorphism, which resulted in differences in AT1 receptor mRNA expression, left ventricular hypertrophy and arterial stiffness between different genetic variants.

Recently, more and more evidences showed that MTHFR 677TT homozygote is associated with increased homocysteine level, especially with low folate level, and this mutation may contribute to early-onset or familial vascular disease. [Deloughery TG et al., Circulation, 1996, 94: 3074-3078]. It was reported that increased homocysteine level is a risk factor for cardio- and cerebrovascular diseases and its sequelae. Putative mechanisms might include endothelial cell injury, endothelial dysfunction, increased vascular smooth muscle cell growth, increased platelet adhesiveness, enhanced LDL oxidation and deposition in the arterial wall, and direct activation of the coagulation cascade. A common genetic mutation in methylenetetrahydrofolate reductase (MTHFR), an enzyme required for efficient homocysteine metabolism, creates a thermolabile enzyme with reduced activity.

To further identify potential effect of Hcy-metablizing enzyme gene, a large-scale cohort study was conducted to elucidate the effect of the MTHFR C677T polymorphism on blood pressure-lowering response induced by ACEI antihypertensive medication [Jiang S et al., Thrombosis Research, 2004, 113: 361-369]. In the study, a total of 444 hypertensive patients, aged 27 to 65 years, were treated orally with Benazepril at a single daily fixed dosage of 10 mg. Results showed that baseline diastolic blood pressure (DBP) and change in diastolic blood pressure (?DBP) were significantly higher in patients with the TT genotype than in those with the CT or CC genotype (P VALUE=0.0076 for DBP, and P VALUE=0.0005 for ? DBP). The finding suggests that MTHFR C677T polymorphism affects the baseline DBP and change in DBP in response to the treatment of ACE inhibitor in Chinese essential hypertensive patients.

Pharmacogenomics is a science that aims to resolve clinical problem of drug efficacy and safety, by identifying polymorphic susceptibility loci across genomic DNA sequences that are associated with drug-specific sensitivity and resistence in patients, and that can accurately predict certain therapeutic response to given drug treatment in specific patient. It is a promising approach to provide an optimal individualized treatment regimen, in order to improve therapeutic efficacy, reduce adverse reactions, and lower medical cost. Its significance in medical care, social wellbeing, and health economics can not be overemphasized.

At present, usage of gene polymorphism in predicting disease risk and therapeutic efficacy is an emerging field. An example of such kind of application is a patent involved in a pre-pregnancy gene diagnostic kit. This patent was applied on Apr. 2, 2001. Its application number is 01107305.5 and authorized public number is CN1155722C. This invention contained methylenetetrahydrofolate reductase (MTHFR) gene and methionine sythase reductase (MTRR) gene. The genetic variants include C677T and A66G located in MTHFR and MTRR genes, respectively. The kit can be used for pre-pregnancy diagnosis and predicting the occurring risk of neural tube abnormity in offspring.

In summary, there are convincing evidence that elevated blood homocysteine level is associated with increased risk and worse prognosis of cerebral and cardiovascular diseases. In order to early detect and prevent cerebrocardiovascular diseases and complications, it is important and necessary to establish a practical and reliable method for predicting individual Hcy level. Previous and our data have also provided convincing evidence that MTHFR gene polymorphisms can predict individual homocysteine level, predict risk and prognosis of cerebral and cardiovascular diseases, and drug treatment effects. At present, anti-hypertensive drugs are prescribed on “a trial and error” basis. Due to the considerable individual variation in drug treatment responses, there is an important need for a kit that will enable clinicians to assess individual risk and select the right type of antihypertensive drugs for the individual, with the goal to achieve high therapeutic efficacy and safety, and to minimize side effects of the drug treatment.

### SUMMARY OF THE INVENTION

Hyperhomocysteinemia is an established risk factor of cerebral and cardiovascular diseases and related complications. Previous and our data have also provided convincing evidence that MTHFR gene polymorphisms can predict individual homocysteine level, and predict risk and prognosis of cerebral and cardiovascular diseases, as well as predict drug treatment effects. In order to realize early detection and prevention of cerebral and cardiovascular diseases and complications, it is important to establish a practical and reliable method for predicting individual Hcy level. Furthermore, it is important to establish a practical and reliable method for predicting individual risk and prognosis of cerebral and cardiovascular diseases, as well as drug treatment effects.

This invention features our discovery on usages of Methylenetetrahydrofolate Reductase (MTHFR) gene polymorphisms in predicting homocysteine (Hcy) level and/or incidence and prognosis of diseases associated with increased Hcy level in a subject, as well as predicting treatment effects of medicines in the category of Angiotension Converting Enzyme Inhibihor (ACEI) with and without combination with B Vitamins. The invention also features our discovery on laboratory and analytical methods that are essential to the above described usages of MTHFR gene polymorphisms. In addition, this invention features a kit that has translated the above discoveries into a practical and reliable tool that can be applied to accomplish the above described usages of MTHFR gene polymorphisms. This invention represents an important step in realizing personalized medicine, with the goal to tailor diagnosis, prevention and treatment strategy to meet individual needs rather than “one size shoe for everyone”.

### DETAILED DESCRIPTION OF THE INVENTION

This invention features our discoveries on usages of Methylenetetrahydrofolate reductase (MTHFR) gene polymorphisms in predicting homocysteine (Hcy) level and/or incidence and prognosis of diseases associated with increased Hcy level in a subject.

The usages of the above described invention, wherein said MTHFR gene polymorphism include at least C677T single nucleotide polymorphism (SNP). Furthermore, SNPs selected from A1298C, G1793A, G215A, G482A and A1317G polymorphisms may be included in the present invention, as well as gene polymorphisms in linkage disequilibrium with the above SNPs, including non-sense mutation, mis-sense mutation and mutation located in the intron or regulation site.

The usages of present invention, wherein said Hcy-associated diseases include but not limited to atherosclerosis, coronary heart disease, cerebrovascular disease, impaired renal function, peripheral vascular disease, arteriovenous thrombosis disease, hypertension, dyslipidemia (abnormal serum lipids), diabetes, psychiosis, cardiovascular diseases, wherein the cardiovascular diseases are preferable. In the present invention, the cardiovascular diseases include but not limited to: acute cardiobrovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, diaphragmatic angina, acute myocardial infarction, cardiac shock, and cardiogenic sudden death. Of which, stroke is the preferred disease. The risk of the mortality from the above diseases is referred as prognosis. In details, wherein
(1) the TT genotype of MTHFR gene C677T polymorphism predicts an increased level of Hcy and an increased risk for hyperhomocysteinemia (high Hcy level);
(2) the CC genotype of MTHFR gene C677T polymorphism predicts a lower level of Hcy and a lower risk for hyperhomocysteinemia (high Hcy level);
(3) the TT genotype of MTHFR gene C677T polymorphism predicts an increased risk of developing Hcy-associated diseases and worse prognosis of theses diseases;
(4) the CC and/or CT genotypes of MTHFR gene C677T polymorphism predicts an decreased risk of developing Hcy-associated diseases and better prognosis of theses diseases.

The usages of the present invention, wherein said subject is preferred to subject with hypertension and/or with hyperhomocystinemia. The invention features on the joint or synergistic effects of MTHFR gene polymorphisms and hypertension and/or increased Hcy level in predicting incident risk and prognosis of Hcy-associated diseases. Among subjects with moderately elevated Hcy level, such synergistic effects of MTHFR gene polymorphisms and hypertension and/or increased Hcy level is more pronounced. Furthermore, wherein said subject with increased Hcy level and with hypertension predict greater risk for Hcy-associated diseases and worse prognosis of these diseases. In the usage of the present invention, where said Hcy-associated diseases include not limited to: atherosclerosis, coronary heart disease, cerebrovascular disease, impaired renal function, peripheral vascular disease, arteriovenous thrombosis disease, hypertension, hyperlipidemia, diabetes, psychosis, cardiovascular diseases, wherein the cardiovascular diseases are preferable. In the present invention, the cardiovascular diseases include but not limited to: acute cardiovascular events, acute cerebrovascular events, cerbral infarction, cerebral hemorrhage, transient cerebral ischemic attack, transient cerebral ischemic, cerebral apoplexy, stroke, acute cardiovascular events, coronary heart disease, diaphragmatic angina, acute myocardial infarction, cardiac shock, and cardiogenic sudden death. Of which, the preferred disease is stroke. The death caused by the above diseases is referred as prognosis. In details, wherein,
(1) the TT genotype of MTHFR C677T polymorphism predicts an increased risk of developing Hcy-associated diseases and a worse prognosis of these diseases in a subject with hyperhomocystinemia (high Hcy level);
(2) the CT or CC genotype of MTHFR C677T polymorphism predicts a lower risk of developing Hcy-associated diseases and better prognosis of these diseases in a subject with hyperhomocystinemia (high Hcy level);
(3) the TT genotype of MTHFR C677T polymorphism predicts an increased risk for the Hcy-associated diseases and a worse prognosis of these diseases in a subject with hyperhomocystinemia (high Hcy level) and hypertension;
(4) the CT or CC genotype of MTHFR gene C677T polymorphism predicts lower risk for the Hcy-associated diseases and better prognosis of these diseases in a subject with hyperhomocystinemia (high Hcy level) and hypertension.

The above described usage of this invention is preferred among those with moderately elevated Hcy level, in particular among those with the level of blood Hcy<=20 umol/L, or below the 90 percentile of the blood Hcy in the population.

This invention features the oligonucleotide fragment which can be used to determine the genotypes of MTHFR gene polymorphism. Preferably, wherein said oligonucleotide fragment is a gene-specified primer or an allele-specified oligonucleotide probe which can be used to determine the genotypes of MTHFR gene polymorphism. Preferably, the oligonucleotide primer or probe is 15-50 bases long.

The selected SNPs of MTHFR gene featured in the present invention include the C677T polymorphism at least. Furthermore, the SNPs selected from A1298C, G1793A, G215A, G482A and A1317G may also be included in the present invention, as well as polymorphisms that are in linkage disequilibrium with the above MTHFR gene polymorphisms, including non-sense mutation, mis-sense mutation and mutations located in the intron or regulation site.

This invention features our discovery on laboratory methods in determining the genotype of the MTHFR gene polymorphisms in a subject, wherein said the selected SNPs of MTHFR gene including the C677T Polymorphism at least, said method comprising the steps of (a) determing genotypes of MTHFR gene SNPs by the above described oligonucleotide fragments; (b) predicting individual Hcy level and /or the development and prognosis of Hcy-associated diseases by usage of genotypes of MTHFR gene polymorphisms.

The method of the present invention, wherein said the selected MTHFR gene polymorphisms include the SNPs selected from A1298C, G1793A, G215A, G482A and A1317G and other polymorphisms that are in linkage disequilibrium with the above SNPs.

The method of the present invention, wherein said Hcy-associated diseases include but no limited to: atherosclerosis, coronary heart disease, cerebrovascular disease, impaired renal function, peripheral vascular disease, arteriovenous thrombosis disease, hypertension, dyslipidemia (abnormal serum lipids), diabetes, psychosis, cardiovascular diseases, wherein the cardiovascular diseases are preferable. In the present invention, the cardiovascular diseases include but not limited to: acute cardiovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, diaphragmatic angina, acute myocardial infarction, cardiac shock, cardiogenic sudden death, the preferred disease is stroke. The Hcy-associated diseases are not limited to the above diseases. Death caused by the above diseases is referred as prognosis. In detail, wherein,
(1) the TT genotype of MTHFR gene C677T polymorphism predicts a higher level of Hcy and an increased risk of hyperhomocysteinemia (high Hcy level);
(2) the CC and/or CT genotype of MTHFR gene C677T polymorphism predicts a lower level of Hcy and a lower risk of hyperhomocysteinemia (high Hcy level);
(3) the TT genotype of MTHFR gene C677T polymorphism predicts a higher risk of developing Hcy-associated diseases and a worse prognosis of theses diseases;
(4) the CC and/or CT genotypes of MTHFR gene C677T polymorphism predicts a lower risk of developing Hcy-associated diseases and better prognosis of theses diseases.

The method of present invention, wherein said subject is preferred to be the subjects with hypertension and/or with hyperhomocystinemia. The invention features on joint and synergistic effects of MTHFR gene polymorphisms and hypertension and/or increased Hcy level on predicting risk and prognosis of Hcy-associated diseases. Such joint and synergistic effects of MTHFR gene polymorphisms and hypertension and/or increased Hcy level is more pronounced among subjects with moderately increased Hcy level, thus, permitting better prediction.

In the method of the present invention, Hcy-associated diseases include but not limited to atherosclerosis, coronary heart disease, cerebrovascular disease, impaired renal function, peripheral vascular disease, arteriovenous thrombosis disease, hypertension, dyslipidemia (abnormal serum lipids), diabetes, psychosis, cardiovascular diseases, wherein the cardiovascular diseases are preferable. In the present invention, the cardiovascular diseases include but not limited to: acute cardio-cerebrovascular events, acute cerebrovascular events, cerbral infarction, cerebral hemorrhage, transient cerebral ischemic attack;transient cerebral ischemic, cerebral apoplexy, stroke, acute cardiovascular events, coronary heart disease, diaphragmatic angina, acute myocardial infarction, cardiac shock, and cardiogenic sudden death. Cardiovascular diseases are preferred Hcy-associated diseases, and stroke is the preferred cardiovascular diseases. Death caused by the above diseases is referred as prognosis. In detail, wherein,
(1) the TT genotype of MTHFR gene C677T polymorphism predicts an increased level of Hcy and an increased risk for hyperhomocysteinemia (high Hcy level);
(2) the CC genotype of MTHFR gene C677T polymorphism predicts a lower level of Hcy and a lower risk for hyperhomocysteinemia (high Hcy level);
(3) the TT genotype of MTHFR gene C677T polymorphism predicts an increased risk of developing Hcy-associated diseases and a worse prognosis of theses diseases;
(4) the CC and/or CT genotypes of MTHFR gene C677T polymorphism predicts an decreased risk of developing Hcy-associated diseases and better prognosis of theses diseases.

In the method of the above described invention, subjects with moderate elevation in Hcy level is preferred, especially as the level of blood Hcy<=20 umol/L, or is below of the 90th percentile of the level of blood Hcy in population. In another word, the prediction performs better in the subjects with moderately elevated Hcy level.

The method of the present invention, wherein said method may be selected from all kinds of nucleic acid analytical techniques as follows: polymerase chain reaction(PCR), polymerase chain reaction-restriction fragment length polymorphism(PCR-RFLP), PCR-allele specificity oligonucleotide probe (PCR-ASO), PCR-sequence specificity oligonucleotide (PCR-SSO), sequencing, PCR-sequence specificity primer (PCR-SSP), PCR-fluorometric method, PCR-finger-printing method, oligonucleotide ligation analysis, fluorescence energy resonance transfer detection, biochip, nucleic acid-chip, DNA-chip, mass spectrum, gene-scan, single strand conformation polymorphism (SSCP), denaturing gel gradient electrophoresis, enzyme or chemistry mismatch cutting method and Taqman method.

In the present invention, wherein said methods such as PCR, PCR-RFLP, biochip, sequencing, gene-scan are the common assay techniques for SNPs genotyping. Taqman technique is a fluorescent real-time quantitative PCR method using allele specificity probes. Biochip technique applys microamount spotting or in situ synthesis to solidify quantity of the biomacromolecular (such as nucleic acid fragments, polypeptide molecular etc) to the surface of various carrier substances (such as glass, slice of silicon, polyacrylamide gel, nylon membrane etc) to compose intensive 2-dimentional arrangement. The labeled target molecules of biological specimen hybrid with the biochip, then the hybridization signals are scanned and analyzed by special instruments (such as scan or electric charge couple photograph camera etc) and the quantity or quality of the target molecular in the sample are determined.

The method of the present invention, wherein the preferred assays among said techniques are PCR, PCR-RFLP, Taqman technique, biochip, gene chip or kits; said the SNPs assays are not the only limited explanation of the genotyping assays. Said methods also include all other regular biotechnology methods of being employed by technologists in the field to determine the SNPs genotypes in the present invention in predicting blood Hcy level or Hcy-associated diseases, as well as other regular biotechnology methods employed to determine the transcription products and/or expression products of SNPs genotypes in the invention in predicting blood Hcy level or Hcy-associated diseases.

The method of the present invention, wherein said biological specimen in a subject can be blood sample, body fluid sample, tissue sample, organ sample, or cultured cells. Blood sample is preferred biological specimen. Wherein said blood sample includes whole blood cells, peripheral blood cells, leucocytes and serum; said body fluid samples include urine, saliva, tissue fluid, cerebrospinal fluid and body cavity efflusion; said tissue samples include oral mucosa swab, hairs, skin, biopsy tissue, tissue similar secretion, excreta samples and so on.

The present invention also features our discovery on a kit which is used in predicting Hcy levels and/or the risk of development and prognosis of the Hcy-associated diseases in a subject using the genotypes of MTHFR gene SNPs.

The selected SNPs of MTHFR gene in the present invention include C677T Polymorphism at least. Wherein said Hcy-associated diseases include but no limited to: atherosclerosis, coronary heart disease, cerebrovascular disease, impaired renal function, peripheral vascular disease, Arteriovenous thrombosis disease, hypertension, hyperlipidemia, diabetes, psychosis, cardiovascular diseases, wherein the cardiovascular diseases are preferable. In the present invention, the cardiovascular diseases include but not limited to: acute cardiovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, pectoris angina, acute myocardial infarction, cardiac shock, sudden death, and cardiogenic sudden death, the preferred disease is stroke. Death caused by the above diseases is referred as prognosis. Said kit of the present invention includes no less than one kind of oligonucleitide fragment in genotyping in the present invention, as well as a selected suitable assay buffer system and color system. In details, wherein
(1) the TT genotype of MTHFR gene C677T polymorphism predicts an increased level of Hcy and an increased risk for hyperhomocysteinemia (high Hcy level);
(2) the CC genotype of MTHFR gene C677T polymorphism predicts a lower level of Hcy and a lower risk for hyperhomocysteinemia;
(3) the TT genotype of MTHFR gene C677T polymorphism predicts an increased risk of developing Hcy-associated diseases and a worse prognosis of theses diseases;
(4) the CC and/or CT genotypes of MTHFR gene C677T polymorphism predicts a lower risk of developing Hcy-associated diseases and better prognosis of theses diseases.

The kit of the present invention, wherein said subject refers to the subject with hypertension or with hyperhomocystinemia; said subject refers to the subject with hypertension and with hyperhomocystinemia.

In the kit described above, moderately elevated Hcy level is preferred, especially as the level of blood Hcy<=20 umol/L, or is below of the 90th percentile of the blood Hcy in the population. In another word, wherein said the method predicts better among subjects with moderately elevated Hcy level.

The kit of the present invention, wherein said the selected SNPs of MTHFR gene include the C677T Polymorphism at least; MTHFR gene polymorphisms also include the SNPs selected form A1298C, G1793A, G215A, G482A, and A1317G and any other polymorphisms that are in linkage disequilibrium with the above SNPs.

The methods and kit of the present invention, wherein said biological specimen can be selected from any kind of blood sample, body fluid sample, tissue sample, organ sample and cultured cells. Blood sample is preferred biological specimen. Wherein said blood samples include whole blood cells, peripheral blood cells, leucocytes and serum; said body fluid samples include urine, saliva, tissue fluid, cerebrospinal fluid and body cavity effusion; said tissue samples include oral mucosa swab, hairs, skin, biopsy tissue, tissue similar secretion, excreta samples.

The kit of the present invention wherein said it comprises regular components, reagents, buffer solution which will be used for the PCR amplification or the biochip, micro-assay system besides the special primers for the forgoing SNPs genotyping. Technologists are expected to be familiar with the above described components and assay methods.

The kit in the present invention wherein said all kinds of the diagnostic reagents and kits based on the prediction methods of the present invention will be included. Based on the MTHFR gene polyrnorphism, various diagnostic reagents and kits may be designed to predict Hcy level and/or the development and prognosis of Hcy-associated disease in a subject.

The usage and method of the present invention are the scientific basis for the validity of our kit. The kit was developed based our findings from our many years of clinical epidemiologic studies that established the association of MTHFR gene polymorphisms with Hcy level, as well as the essential laboratory and analytical methods we developed. The epidemiology studies include at least the prospective nest case-control study, in which we also established associations of MTHFR gene SNPs, hypertension, Hcy level with the risk and prognosis of cerebral and cardiovascular events, with concurrent measures blood pressure, Hcy level, and other epidemiological and clinical variables.

The present invention features our discoveries on usage of MTHFR gene polymorphism in predicting treatment effect of ACEI medicine in a subject, said MTHFR gene polymorphisms including the C677T and/or A1298C polymorphism at least, said treatment effect including increased Hcy level, and impaired liver function.

The usage of MTHFR gene polymorphisms in predicting treatment effect, wherein said MTHFR gene polymorphisms also including the SNPs selected from G1793A, G215A, G482A, and A1317G and any other polymorphisms that are in linkage disequilibrium with the above SNPs.

In an embodiment of the present invention in predicting treatment effect of ACEI medicine in a subject, wherein said ACEI medicine including angiotensin converting enzyme inhibitors (ACEI), said treatment effect including increased Hcy level. In another embodiment of the present invention in predicting treatment effect, wherein said ACEI medicine including including ACEI, said treatment effect including liver injury as measured by elevated liver enzymes (ALT, AST).

In details of explanation, ACEI modulates blood pressure by affecting renin-angiotension-aldosterone (RAAS), where ACEI can modulate blood pressure by two ways: (a) ACEI inhibits activity of angiotensin converting enzyme (ACE) by combining angiotensin converting enzyme I, inhibits the production of angiotensin II and decreases secretion of aldosterone; (b) ACEI inhibits decomposition of bradykinin, activates NO synthesis (NOS) to protect the vascular endothelial cells and decrease the vascular tension caused by sympathetic nerve.

In the usage of the present invention, wherein said ACEI medicine are in the category of Angiotension Converting Enzyme Inhibihor (ACEI), which include but not limited to benazepril, captopril, enalapril, cilazapril, peridopril, delapril, quinapril, lisinopril, ramipril, imidapril, zofenopril, trandolapril and fosinopril, of which the preferred medication is enapril, benazepril, lisinopril or fosinopril. In details, wherein
(1) subjects with the MTHFR 677TT homozygote genotype and/or 1298AA homozygote genotype tend to have greater increase in Hcy level induced by the treatment of ACEI medicine, while subjects with the MTHFR 677CC homozygote genotype and/or 1298CC homozygote genotype tend to have smaller increase in Hcy level induced by the treatment of ACEI medicine.
(2) subjects with the MTHFR 677CC homozygote genotype tend to have greater impairment in liver function induced by the treatment of ACEI medicine; while subjects with the MTHFR 677TT homozygote genotype tend to have less impairement in liver function induced by the treatment of ACEI medicine.

This invention features a usage of MTHFR gene polymorphisms in predicting treatment effects of medical compounds in a subject: said MTHFR gene polymorphisms including the C677T and/or A1298C polymorphism at least; said medical compounds including ACEI medicines and B vitamins; said treatment effects include (1) lowering Hcy level; (2) reducing liver injury as measured by elevated ALT and AST induced by ACEI medicine; (3) lowering blood pressure; and/or (4) protecting target organs such as kidney.

The usage of the present invention, wherein said MTHFR gene polymorphisms also include the SNPs selected from G1793A, G215A, G482A, and A1317G and any other polymorphisms that are in linkage disequilibrium with the above SNPs.

In details, the usage of the present invention, wherein said medical compounds comprise ACEI medicines and B vitamins; said ACEI medicines include but not limited to benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinopril; said B vitamins include folic acid and its analogues, vitamin B6 and vitamin B12, of which the preferred B vitamin is folic acid. Said the folic acid and its analogues include 5-formyl-tetrahydrofolates, calcium leucovorin, metafolin, folic acid salt, active metabolism product of folic acid or folic acid salt, substance which can release or produce folic acid. Further, wherein said medical compounds are compounds comprising enapril and folic acid or comprising benazepril and folic acid.

In details of the usage of the present invention, wherein
(1) the MTHFR 677TT homozygote genotype and/or 1298AA homozygote genotype predicts that said medical compound is likely to have a greater effect in lowering Hcy level; while the MTHFR 677CC homozygote genotype and/or 1298CC homozygote genotype predicts that said medical compound is likely to have a weaker effect in lowering Hcy level;
(2) the MTHFR 677CC homozygote genotype predicts that said medical compound is likely to have a greater effect in reducing liver function damage induced by ACEI medicine; while the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure; while the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs, while the MTHFR 677CC homozygote genotype and/or 1298CC homozygote genotype predicts that said medical compound is likely to have a weaker effect in protecting target organs.

The usage of the present invention, wherein said “protecting target organs” includes protection of renal function, prevention of re-stenosis after percutaneous transluminal coronary angioplasty (PTCA), prevention from hypertension and cardiovascular or cerebrovascular diseases associated complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, retinal artery sclerosis, retinal artery occulation, arteriosclerotic retinopathy, and arteriosclerotic retinitis.

Another aspect of the present invention features the oligonucleotide fragment which can be used to determine the genotypes of MTHFR gene polymorphisms. Wherein said oligonucleotide fragment is (1) a gene specific primer or (2) an allele specific oligonucleotide probe which can be used to determine the genotypes of MTHFR gene polymorphisms. Wherein said oligonucleotide fragment is preferable 10-50 nucleotides long.

In the present invention, the selected SNPs of MTHFR gene include the C677T and/or A1298C polymorphism at least. Furthermore, the G1793A, G215A, G482A and A1317G polymorphisms may also be included, as well as gene polymorphisms in linkage disequilibrium with the above SNPs of MTHFR gene polymorphism, including no-sense mutation, mis-sense mutation, mutation located in intron or regulation site of MTHFR gene.

This invention features the methods in predicting the treatment effect of medicine in a subject using MTHFR gene polymorphisms, wherein said MTHFR gene polymorphisms including the C677T and/or A1298C polymorphism at least, said medicine including ACEI medicine, said treatment effect including increased Hcy level and damage to liver function, said method comprising the following steps of (a) determining the genotypes of MTHFR gene polymorphisms by the oligonucleotide fragment described above; (b) predicting the treatment effects of said ACEI medicines using the genotypes of MTHFR gene polymorphisms.

In the method of the present invention, wherein said MTHFR gene polymorphisms also can be selected from G1793A, G215A, G482A and A1317G. Additionally, the selected MTHFR gene polymorphisms may involve the polymorphisms that are in linkage disequilibrium with the above SNPs.

In another embodiment of said method, the said medicine is angiotensin converting enzyme inhibitor (ACEI), said treatment effect is elevated Hcy level. In another embodiment of said method, the said medicine is ACEI, said treatment effect is liver injury as measured by elevated liver enzymes ALT and AST. In details, wherein said ACEI medicine include benazepril, captopril, enalapril, cilazapril, peridopril, delapril, quinapril, lisinopril, ramipril, imidapril, zofenopril, trandolapril and fosinopril, of which the preferred ACEI medicine is Enapril, benazepril, lisinopril or fosinopril.

The method of the present invention in predicting treatment effect of ACEI medicine, wherein
(1) subjects with the MTHFR 677TT homozygote genotype and/or 1298AA homozygote genotype tend to have greater increase in Hcy level induced by the treatment of ACEI medicine, subjects with the MTHFR 677CC homozygote genotype and/or 1298CC homozygote genotype tend to have smaller increase in Hcy level induced by the treatment of ACEI medicine.
(2) subjects with the MTHFR 677CC homozygote genotype tend to have greater impairment in liver function induced by the treatment of ACEI medicine; subjects with the MTHFR 677TT homozygote genotype tend to have less impairment in liver function induced by the treatment of ACEI medicine.

In another embodiment, the medical compounds comprising ACEI medicine and B vitamins, said treatment effects are: (1) lowering Hcy level, (2) reducing damage to liver function induced by ACEI medicine, (3) lowering blood pressure, and/or (4) protecting target organs.

In the present invention, wherein said ACEI medicines can be selected from benazepril, captopril, enalapril, delapril, quinapril, lisinopril, ramipril, imidapril, zofenopril, trandolapril and fosinopril. Among these ACEI medicines, enapril, benazepril, lisinopril and fosinopril are preferred ACEI medicines. In the present invention, wherein said medical compounds are compounds comprising ACEI medicine and B vitamins, said B vitamins can be selected from folic acid and its analogues, vitamin B6 and vitamin B 12. Wherein said folic acid analogues include 5-formyl-tetrahydrofolates, calcium leucovorin, metafolin, folic acid salt, and active metabolism product of folic acid or folic acid salt, substance which can release or produce folic acid. Further, wherein said medical compounds prefers the compounds comprising enapril and folic acid or comprising benazepril and folic acid.

In details, the method of wherein
(1) the MTHFR 677TT homozygote genotype and/or 1298AA homozygote genotype predicts that said medical compound is likely to have a greater effect in lowering Hcy level; while the MTHFR 677CC homozygote genotype and/or 1298CC homozygote genotype predicts that said medical compound is likely to have a weaker effect in lowering Hcy level;
(2) the MTHFR 677CC homozygote genotype predicts that said medical compound is likely to have a stronger effect in reducing liver function damage induced by ACEI medicine; while the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure; while the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs; while the MTHFR 677CC homozygote genotype and/or 1298CC homozygote genotype predicts that said medical compound is likely to have a weaker effect in protecting target organs.

In the present invention, wherein said “target organ protection” include protection of renal function, prevention of restenosis after percutaneous transluminal coronary angioplasty (PTCA) and prevention from hypertension and cardiovascular or cerebrovascular diseases complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, uetinal artery sclerosis, uetinal artery occulation, arteriosclerotic retinopathy, and arteriosclerotic retinitis.

The methods of the present invention, wherein said method can be selected from any kind of nucleic acid analytical techniques as follows: polymerase chain reaction(PCR), polymerase chain reaction-restriction fragment length polymorphism (PCR-RFLP), PCR-allele specificity oligonucleotide probe (PCR-ASO), PCR-sequence specificity oligonucleotide (PCR-SSO), sequencing, PCR-sequence specificity primer (PCR-SSP), PCR-fluorometric method, PCR-finger-printing method, oligonucleotide ligation analysis, fluorescence energy resonance transfer detection, biochip, nucleic acid-chip, DNA-chip, mass spectrum, gene-scan, single strand conformation polymorphism (SSCP), denaturing gel gradient electrophoresis, enzyme or chemistry mismatch cutting method and Taqman method.

In the present invention, wherein said PCR, PCR-RFLP, biochip, sequencing, gene-scan and so on, these method are the common assay technique for SNPs genotyping. Taqman technique is a kind of fluorescent real-time quantitative PCR method using allele specificity probes. Biochip technique apply microamount spotting or in situ synthesis to solidify quantity of the biomacromolecular (such as nucleic acid fragments, polypeptide molecular etc) to the surface of various carrier substances(such as glass, slice of silicon, polyacrylamide gel, nylon membrane etc) to compose intensive 2-dimentional arrangement. The labeled target molecules of biological specimen hybrid with the biochip, then the hybridization signals are scanned and analyzed by special instruments (such as scan or electric charge couple photograph camera etc) and the quantity or quality of the target molecular in the sample are determined.

The method of the present invention, wherein said PCR, PCR-RFLP, Taqman technique, biochip, gene chip or kits are preferred assays among the above techniques. The explanation about the SNPs assays are not limited to the genotyping assays. Said methods also include all other regular biotechnology methods employed by technologists in this field to determine the SNPs genotypes of the present invention to predict the treatment effect of ACEI medicine or ACEI medical compounds, as well as the other regular biotechnology methods employed to determine the transcription products and/or expression products of SNPs genotypes of the invention to predict the treatment effect of medical compounds comprising of ACEI.

In the method of the present invention, wherein said biological specimen from a subject includes blood sample such as whole blood cells, peripheral hematocytes, white blood cells; body fluid sample such as saliva, urine, seminal fluid, serum, plasma; tissue sample such as oral mucosal epithelia cells, hair, skin, biopsy samples; tissue-like secretion; excretion sample; culture cells. Herein said blood sample is preferred biological specimen. Said sample may be purified, such as separation of the whole nucleic acid.

The present invention also features our discovery on a kit which is used in predicting treatment effect of medicine in a subject using MTHFR gene polymorphism, said MTHFR gene polymorphisms include at least C677T and/or A1298C SNP, said medicine including ACEI medicines, said treatment effects include increased Hcy level and damage to liver function induced by the ACEI medicine, said kit including no less than one kind of the above said oligonucleotide fragment, and suitable assay buffer system and color system adaptable to the detection. The kit of the present invention, wherein said MTHFR gene polymorphisms also include the SNPs selected from G1793A, G215A, G482A, and A1317G and any other polymorphisms that are in linkage disequilibrium with the above SNPs.

The kit of the present invention, wherein said ACEI medicines include benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinopril. In details, wherein said
(1) subjects with the MTHFR 677TT homozygote genotype and/or 1298AA homozygote genotype tend to have greater increase in Hcy level induced by the treatment of ACEI medicine, subjects with the MTHFR 677CC homozygote genotype and/or 1298CC homozygote genotype tend to have smaller increase in Hcy level induced by the treatment of ACEI medicine;
(2) subjects with the MTHFR 677CC homozygote genotype tend to have greater impairment in liver function induced by the treatment of ACEI medicine; subjects with the MTHFR 677TT homozygote genotype tend to have less impairment in liver function induced by the treatment of ACEI medicine.

Another aspect of the invention features a kit of predicting the treatment effect of medical compounds in a subject using MTHFR gene polymorphisms, said medical compounds including ACEI medicine and B vitamins, said treatment effect including (1) reducing the Hcy level; (2) reducing liver damage induced by ACEI medicine; (3) lowering blood pressure; and/or (4) protecting target organ, said MTHFR gene polymorphisms including the C677T and/or A1298C polymorphism at least, said kit includes no less than one oligonucleotide fragment in the present invention, and suitable assay buffer system and color system adaptable to the detection.

In detail, wherein

(1) the MTHFR 677TT homozygote genotype and/or 1298AA homozygote genotype predicts that said medical compound is likely to have a greater effect in lowering Hcy level; while the MTHFR 677CC homozygote genotype and/or 1298CC homozygote genotype predicts that said medical compound is likely to have a weaker effect in lowering Hcy level;
(2) the MTHFR 677CC homozygote genotype predicts that said medical compound is likely to have a greater effect in reducing liver function damage induced by ACEI medicine, the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure, while the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs, while the MTHFR 677CC homozygote genotype and/or 1298CC homozygote genotype predicts that said medical compound is likely to have a weaker effect in protecting target organs.

The kit of the present invetion, wherein said “protecting target organ” includes protection of renal function, prevention from re-stenosis after percutaneous transluminal coronary angioplasty (PTCA), and prevention from hypertension and cardiovascular or cerebrovascular diseases associated complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, retinal artery sclerosis, retinal artery occulation, arteriosclerotic retinopathy, and arteriosclerotic retinitis.

In the method of the present invention, wherein said MTHFR gene polymorphisms also can be selected from G1793A, G215A, G482A and A1317G polymorphisms. Additionally, the selected MTHFR gene polymorphisms may include those in linkage disequilibrium with the above SNPs of MTHFR gene polymorphisms, including no-sense mutation, mis-sense mutation, mutation located in intron or regulation site of MTHFR gene.

A gene chip that includes the oligonucleotide fragment in the present invention for determining the genotypes of MTHFR gene polymorphisms.

### BACKGROUND OF THE PRESENT INVENTION

The present invention is based on our many years of clinical epidemiological and pharmacogenomic studies which recruited over 30,000 subjects. Hypertensive subjects were treated with ACEI medicines or ACEI medicine combined with B-vitamins. Specifically, Enalapril is one of the representative ACEI medicines, which converts to enalaprilat by hydrolysis metabolism in liver. ACEI inhibit the activity of angiotensin converting enzyme (ACE) by combining with angiotensin converting enzyme I to inhibit the production of angiotensin II, so to increase the activity of rein and decrease secretion of aldosterone and lower the vessel tension. Enalapril can interfere with the decomposition of bradykinin, activate NO synthesis (NOS) to protect the vascular endothelial cells and decrease the vascular tension caused by sympathetic nerve. Enalaprilat, the metabolism product of enalapril, has powerful effect on inhibiting angiotensin converting enzyme I than enalapril. The half-time of enalapril is 11 hours and the blood pressure lowering effect may last for more than 24 hours with the regular dosage. The regular dosage for lowering blood pressure is 5 mg, QD and may adjust to 10˜40 mg per day based on blood pressure response.

Human MTHFR gene is located at chromosome 1p36.3, the gene include exon 11 and intron 10, the length distribution of the exons is 99-252 bp, that of the introns is 192-981 bp. Full-Length cDNA coding for a flavoprotein is 2.2 kb, of which biochemistry function is to catalyze reduction reaction of 5,10-methene-tetra-hydrofolic acid to 5-methyl-tetra-hydrofolic acid [Zhou J, Kang S S, Wong P W K, et al. Purification and characterization of methylenetetrahydrofolate reductase from human cadaver liver. Biochem Med Metab Bio, 1990, 43: 234-242].

5-methyl-tetra-hydrofolic acid is a kind of methyl donor, participating in many important bioprocess (eg. synthesis of purine and pyrimidine). Besides, it is related to blanket methylation of homocysteic acid in the methionine metabolism. MTHFR polymorphic site genotype can alter MTHFR function activity. MTHFR enzyme activity degradation will lead to accumulation of HCY, which is considered a risk factor for cardiovascular disease and may also be considered to be potential teratogenic and toxic effects on embryo and fetus. 5,10-Methylenetetrahydrofolate reductase (MTHFR) gene defect is associated with occurrence of cardiovascular disease and congenital malformations.

Hyperhomocysteinemia can facilitate development and occurrence of all important cardiovascular and cerebrovascular diseases, including coronary artery disease, cerebral apoplexy, renal function lesion, periphery artery disease etc. Higher HCY level may be an important factor that lead to target organ damage and loss, eg, heart, brain, kidney etc in hypertensive patients. Frequent SNP situs can be located in the region of exon, intron and noncoding region including regulatory site, optimizing exon, especially polymorphism sites that can alter coding amino acid sequence.

According to the MTHFR polymorphism sites, various kinds of diagnostic reagents and kits to predict the treatment effect of medicines comprising of ACEI can be designed and developed. All kinds of diagnostic reagents and kits based on the predictive approach and usage described in this invention are within the scope and claims of this invention.

The “kit” in the present invention is not restricted to the concrete forms of kit, rather, it may display as microchip, microarray, microdetective system or detective system depending on all kinds of carriers, and include the same package form of said detective system above, such as microplate system, paper carrier, glass carrier, nylon carrier, plastic carrier, silica carrier, gelatin carrier, membrane carrier and so on.

In another related aspect, the present invention features a gene chip or DNA array. The gene chip includes MTHFR polymorphism genotyping oligonucleotide in the present invention. The DNA chip is preferable in the present invention.

Key Research Findings that Led to the Present Invention:

Through our follow-up study of more than 30,000 study subjects for 2-10 years, we prospectively identified 204 patients with cardiovascular and cerebrovascular discrease. Of them, 148 subjects have complete MTHFR C677T genotype data. We identified corresponding controls use 1:1 ratio and matched controls by sex, age, and living region.

By measuring individual HCY density which is divided into different levels and cutoff points, we found that different MTHFR C677T polymorphism genotype site can predict different HCY density level, and possess good positive predictive value and negative predictive value. We also found that the effect of the MTHFR C677T polymorphism on the risk of Hcy associated diseases and prognosis varied by different HCY density levels.

As one of the most significant findings, MTHFR polymorphism genotype, hypertension and blood HCY level have a joint or synergistic effect in predicting occurrence and prognosis of individual cerebrovascular disease. For example, total cardiovascular and cerebrovascular event risk of the hypertension patients with MTHFR 677TT genotype is 6.4 times (2.3-17.6) higher than the patients without hypertension and without 677TT genotype; stroke event risk is 8.2 times (2.7-24.7) higher; cardiovascular and cerebrovascular death risk is 5.3 times (1.7-16.5) higher.

1) After removing the study samples whose serum HCY level is 90% in all control group, the total cardiovascular and cerebrovascular event occurrence risk of the hypertension patients with 677TT genotype is 14.5 times higher (2.8-75.3) than the patients without hypertension and 677TT genotype, stroke event risk of that is 15.5 times (2.8-86.0) higher, death rate risk of that is 26.8 times (3.0-236.3) higher.

After removing the subjects whose serum HCY level is more than 20 μmol/L, the total cardiovascular and cerebrovascular event occurrence risk of the hypertensive patients with 677TT genotype is 15.0 times higher (2.9-77.7) than the patients without hypertension and without 677TT genotype, stroke event risk is 16.0 times (2.9-88.6) higher, death risk is 20.2 times (2.2-187.0) higher.

We conducted a clinical epidemiology study, in which we classified hypertensive subjects into 677CC, 677CT, and 677TT groups based on the genotypes of MTHFR C677T polymorphism. Each subject was treated with Enalapril alone or combined with folate for 57 consecutive days. Pre-treatment and post-treatment plasma Hcy level, blood pressure (SBP/DBP), and urine albumin were measured. The major findings are summarized as follow.

Subjects treated with Enalapril alone for 57 days had an increased level of plasma Hcy. However, the degree of the increase in Hcy level was associated with the genotype of C677T polymorphism. Compared to subjects with 677CT or 677CC genotype, subjects with 677TT genotype had a significantly greater increase in plasma Hcy level post Enalapril treatment. Such finding persisted after adjustment for pertinent covariates. There was no significant difference in the increase of Hcy level between subjects with 677CT and subjects with 677CC genotype. These results suggest an association between MTHFR C677T polymorphism and ENALAPRIL treatment-induced increase in plasma Hcy level, which was particularly pronounced among subjects with MTHFR 677TT genotype.

Another important finding is related to ACEI medicine combined with B-vitamin. For hypertensive patients treated with a combination of ENALAPRIL and folate for 57 consecutive days, the MTHFR C677T polymorphism affects the Hcy-lowering response induced by the treatment. Here, the Hcy-lowering response is defined as 0^{th} day's Hcy level-57^{th} day's Hcy level. The more 677T alleles carried by a subject, the greater the Hcy-lowering response post the treatment. Compared to subjects with 677CC genotype, subjects with 677TT genotype had a significantly greater plasma Hcy-lowering response. However, there was no significant difference between 677CT and 677CC groups. Our data indicate that the MTHFR C677T polymorphism can predict Hcy-lowering response induced by the combination therapy of ENALAPRIL and folate.

Among hypertensive subjects treated with a combination therapy of ENALAPRIL and folate for 57 consecutive days, the MTHFR C677T polymorphism affects its blood pressure-lowering response. The more 677T alleles carried by a subject, the greater the blood pressure-lowering response to the combination therapy. Compared to subjects with 677CC, subjects with 677TT had significantly greater blood pressure-lowing response, which is true for both systolic and diastolic blood pressure. However, there was no significant difference between the 677CT and CC groups. Our data indicate that the MTHFR C677T polymorphism can predict blood pressure-lowering response induced by the combination therapy, and such treatment response is more pronounced among subjects with the MTHFR 677TT genotype.

Among hypertensive subjects treated with combination therapy of ENALAPRIL and folate for 57 consecutive days, their urine albumin level was significantly reduced. Moreover, the MTHFR C677T polymorphism affects this urine albumin reduction effect, which is defined as 0^{th} day's urine albumin level-57^{th} day's urine albumin level. Compared to subjects with 677CC genotype, subjects with 677TT genotype had a significantly greater reduction in urine albumin level, but there is no significant difference between 677CT and 677 CC groups. Our data indicate that the MTHFR C677T polymorphism can predict urine albumin reduction effect induced by the combination therapy.

Among hypertensive subjects treated with ENAZEPRIL combined with folate for 57 consecutive days, their urine albumin level was significantly reduced. Moreover, this treatment induced-urine albubin reduction effect varied by the MTHFR A1298C polymorphism. Compared to subjects with 1298CC genotype, subjects with 1298AA had a more significant reduction of urine albumen level, but no difference between 1298AC and 1298CC groups was observed. Our data indicated that the MTHFR A1298C polymorphism can predict urine albumin reduction response induced by the treatment of ENAZEPRIL combined with folate, and such response is more pronounced among subjects with 1298AA genotype.

In our clinical epidemiology study, we classified subjects into 677CC, 677CT, and 677TT groups based on the MTHFR C677T genotype. Each subject was treated with ENAZEPRIL alone or combined with folate for 57 consecutive days. Pre-treatment and post-treatment liver enzymes (ALT and AST) were measured. The MTHFR C677T polymorphism can affect the degree of liver injury induced by ACEI medicine combined with folate. Controlling for MTHFR C677T genotype, subjects treated with combination of Enazepril and folate had a smaller increase in ALT and AST. Remarkably, given the combination therapy, Subjects with MTHFR 677CC genotype tend to have greater increase in ALT and AST, but subjects with MTHFR 677TT genotype tend to have less increase in ALT and AST. Subjects with MTHFR 677CT genotype fell in-between. Our data indicate that the MTHFR C677T polymorphism and combination of Enazepril with folate can jointly affect the degree of liver injury, in which subjects with MTHFR 677TT genotype and treated with combination of Enazepril with folate showed no elevation in ALT and AST but subjects with MTHFR 677CC genotype and treated with Enazepril alone showed the greatest increase in ALT and AST.

In the present invention, we especially designed primer sequence for analyzing genotypes of MTHFR polymorphism sites, which has the strength of high amplification efficiency, good specificity, time saving, and easy to use. The special primers for the genotype of MTHFR gene C677T polymorphism are shown as follow, and the fragment length obtained by means of amplification was 274 bp:

forward primer: 5′- CTT TGA GGC TGA CCT GAA GC-3′
reverse primer: 5′- CTG GGA AGA ACT GAG CGA AC

### EXAMPLES

### Example 1

Detect MTHFR C677T Polymorphism Genotype (Ala222Val, dsSNP ID: rs1801133) and Predict Homocysteine (Hcy) Level in Plasma.

1.1 Determine MTHFR C677T Polymorphism Genotype.

1) Extract Genomic DNA from a Subject's Whole Blood Sample.

a. Add 30 ml RBC lysis solution to the whole blood sample, mix gently, incubate the sample on RT for 10 minutes and shake several times during incubation to break down RBC completely.
b. Spin the sample for 10 minutes at 2000 rpm using a Beckman JS-5.2 rotor at 4° C. Discard the supernatant and vortex the pellet. Add 40 ul proteinase and 50 ul RNase, mix, add 15 ml WBC lysis solution. Incubate at 37° C. for 20 minutes, chill the tube on ice for 10 minutes.
c. Add 4 ml cold protein precipitated solution, mix and at −20° C. for 5 minutes. Spin the sample for 10 minutes at 3000 rpm using a Beckman JS-5.2 rotor at 4° C. Gently shake several times after adding the supernatant to 15 ml eppendorf tube with 15 ml isopropyl alcohol until forming DNA flocculation precipitation.
d. Transfer the precipitation to another 1.5 ml centrifuge tube, add 1 ml 75% ethanol to wash, air dry.
e. Add 1.0 ml ddH₂O and shake overnight.
f. Detect the DNA concentration by ultraviolet spectrophotometry method. The DNA concentration (ug DNA/ml) is calculated by the formula: (A260 reading)×(50 μg/ml constant)×(Dilution Factor), and the DNA quality is evaluated by the 260/280 ratio, which is ideally at 1.8˜2.0.
   2) Detect MTHFR C677T Polymorphism Site by Polymerase Chain Reaction—Restriction Fragment Length Polymorphism (PCR-RFLP)

Design specific primers for PCR by MTHFR C677T gene sequence including forward primer and backward primer. PCR amplification is performed by routine method as described below.

The primer sequence:

Forward primer: 5′- CTT TGA GGC TGA CCT GAA GC-3′
Backward primer: 5′- CTG GGA AGA ACT CAG CGA AC-3′

Reaction System of PCR:

45 ng Genomic DNA, 10 pmol (20 μmol/L) of each of the two primers, 2.0 mmol/UL dNTPs, 1.0 μl 10× buffer, 3 U Gold Taq DNA polymerase, add ddH2O to 10 μl final volume.

Reaction Condition of PCR:

Predenaturation at 95° C. for 10 minutes; Denaturation at 94° C. for 30 seconds, Anneal primers at 59° C. for 45 seconds, Extend primers at 68° C. for 45 seconds, 35 cycles; extend at 68° C. for 7 minutes, and finally to acquire the 274 bp fragment.

The Condition and System of RFLP:

The reaction volume is 15 μl including 10 μl PCR product, 1.5 μl 10× NEBuffer#2, 4 U Hinf I endonuclease and 3.1 μl ddH2O. Incubate at 37° C. overnight.

The Recognized Site of Hinf I Endonuclease is:

5′ . . . G [symbol] A N T C. . . 3′
3′ . . . C T N A [symbol] G. . . 5′

Determination of MTHFR Genotype:

The products after endonuclease reaction are separated by electrophoresis and read under ultraviolet light. The MTHFR genotype is determined as follows:

If the restriction fragment length is 274 bp, the genotype is 677CC;

If the restriction fragment lengths are 274 bp and 228 bp and 46 bp, the genotype is 677CT;

If the restriction fragment length is 228 bp and 46 bp, the genotype is 677TT.

1.2 Prediction of Plasma Hcy Level

MTHFR 677TT carrier predicts a higher level of plasma Hcy, with a greater probability of elevated Hcy level
MTHFR 677CC or 677CT carriers predict a lower level of plasma Hcy, with a greater probability of decreased Hcy level
MTHFR 677TT carrier predicts a higher risk of Cerebral and cardiovascular diseases.
MTHFR 677CC or 677CT carriers predict a lower risk of Cerebral and cardiovascular diseases.

In our population-based epidemiological study, we classified subjects into 677CC, 677CT, and 677TT groups by the MTHFR C677T genotype. Subjects' plasma Hcy level and basic characteristics such as sex, age, height and weight were also obtained. Table 1 shows the association of baseline Hcy level with MTHFR C677T genotype.

The MTHFR C677T genotype was significantly associated with Hcy level in plasma. Subjects bearing 677TT genotype had a significantly higher Hcy level (Mean±SD: 18.65±11.03 μmol/L) compared to subjects with 677CC or 677CT genotypes (10.74±3.60 μmol/L or 11.19±4.30 μmol/L; respectively). After adjusting for pertinent covariates, the findings persisted (β=9.8, se=0.9, P<0.0001).

TABLE 1
Effect of MTHFR C677T polymorphism on the baseline
homocysteine (Hcy) level in plasma (μmol/L).

| MTHFR | | Hcy | | | |
|---|---|---|---|---|---|
| Genotype | N | (Mean ± SD) | ? | SE | P |
| CC | 337 | 10.74 ± 3.60 | — | — | — |
| CT | 509 | 11.19 ± 4.30 | 1.1 | 0.8 | 0.230 |
| TT | 241 | 18.65 ± 11.03 | 9.8 | 0.9 | 0.000 |

# The multiple linear regression model adjusted for sex, age, height, and weight.

In a case-control study, we evaluated sensitivity, specificity, positive predictive value (PPV), and negative predictive value (NPV) of MTHFR C677T polymorphism (TT vs. CC genotype) in predicting elevated plasma Hcy level, which is defined by three different cutoff points (10 μmol/L, 15 μmol/L, and 20 μmol/L). As shown in TABLE 2, the sensitivity, specificity, PPV, and NPV change with cutoff points: Higher cutoff points are associated with higher sensitivity and NPV, while lower cutoff points are associated with higher PPV. For example, using 20 μmol/L as a cutoff point for elevated Hcy level, its sensitivity is 91.9%, that is, it can correctly identify 91.9% of subjects with elevated Hcy level; and its NPV is 98.3%, that is, it can correctly identify 98.3% of subjects who are predicted to have lower plasma Hcy level. The data in Table 2 indicate that 677TT homozygote genotype can predict elevated levels of Hcy in plasma, while the 677CC homozygote predicts lower levels of Hcy in plasma.

TABLE 2
Sensitivity, specificity, positive predictive value (PPV),
and negative predictive value (NPV) of MTHFR C677T Polymorphism
(TT vs. CC) in predicting elevated plasma homocysteine (Hcy)
level, by three different cutoff points.

| Cutoff point | | | | | | Spec- | | |
|---|---|---|---|---|---|---|---|---|
| defining | 677TT | | 677CC | | Sensi- | ific- | | |
| elevated | High | Low | High | Low | tivity | ity | PPV | NPV |
| Hcy | Hcy | Hcy | Hcy | Hcy | (%) | (%) | (%) | (%) |
| 10(μmol/L) | 185 | 56 | 181 | 156 | 50.5 | 73.6 | 76.8 | 46.3 |
| 15(μmol/L) | 102 | 139 | 36 | 301 | 73.9 | 68.4 | 42.3 | 89.3 |
| 20(μmol/L) | 68 | 173 | 6 | 331 | 91.9 | 69.8 | 28.2 | 98.2 |

TABLE 3
Association of MTHFR C677T polymorphism with the
risk of elevated plasma homocysteine (Hcy) level
defined by three different cutoff points.

| Cutoff points/ | Total | hHcy* | | | | |
|---|---|---|---|---|---|---|
| Genotype | N | N | % | OR# | 95% CI | P |
| =10(μmol/L) | | | | | | |
| CC | 337 | 181 | 53.7 | 1.00 | — | — |
| TT | 241 | 185 | 76.8 | 2.8 | 2.1-4.8 | 0.00 |
| =15(μmol/L) | | | | | | |
| CC | 337 | 36 | 10.7 | 1.00 | — | — |
| TT | 241 | 112 | 46.5 | 6.1 | 4.2-9.7 | 0.00 |
| =20(μmol/L) | | | | | | |
| CC | 337 | 6 | 1.8 | 1.00 | — | — |
| TT | 241 | 68 | 28.2 | 23.5 | 11.3-52.1 | 0.00 |

#The Logistic Regression Models adjusted for sex, age, height, and weight.
*Hyperhomocysteinemia.

We further assessed MTHFR 677TT homozygosity in predicting the risk of elevated Hcy level, compared to 677CC homozygosity, using Logistic Regression analysis with adjustment for the pertinent covariates. As shown in TABLE 3, regardless of the cutoff points, MTHFR 677TT homozygosity is associated with higher risk of elevated plasma Hcy level, and MTHFR 677CC homozygosity is associated with lower risk of elevated plasma Hcy level. Furthermore, the higher the cutoff point, the higher the risk of elevated plasma Hcy level as measured by odd ratio (OR) associated with MTHFR 677TT genotype compared to 677CC genotype. Specifically, the OR is 2.8 (95% CI: 2.1-4.8), 6.1 (95% CI: 4.2-9.7), and 23.5 (95% CI: 11.3-52.1) for the cutoff point of 10, 15, and 20 μmol/L, respectively.

We further evaluated sensitivity, specificity, positive predictive value (PPV), and negative predictive value (NPV) of MTHFR C677T polymorphism (TT vs. CC+CT genotype) in predicting elevated plasma Hcy level, which is defined by three different cutoff points (10 μmol/L, 15 μmol/L, and 20 μmol/L). As shown in TABLE 4, the sensitivity, specificity, PPV, and NPV change with cutoff points: The higher cutoff points are associated with higher sensitivity and NPV, while lower cutoff points are associated with higher PPV. For example, using 20 μmol/L as a cutoff point for elevated Hcy level, its sensitivity is 70.8%; and its NPV is 96.7%. The data in Table 4 indicate that 677TT genotype can predict elevated levels of Hcy in plasma, while the 677CC+CT genotype predicts lower levels of Hcy in plasma.

TABLE 4
Sensitivity, specificity, positive predictive value (PPV),
and negative predictive value (NPV) of MTHFR C677T polymorphism
(TT vs. CC + CT) in predicting elevated plasma homocysteine
(Hcy) level, by three different cutoff points

| Cutoffs | | | | | | Spec- | | |
|---|---|---|---|---|---|---|---|---|
| defining | TT | | CC + CT | | Sensi- | ific- | | |
| elevated | High | Low | High | Low | tivity | ity | PPV | NPV |
| Hcy | Hcy | Hcy | Hcy | Hcy | (%) | (%) | (%) | (%) |
| =10(μmol/L) | 185 | 56 | 461 | 385 | 28.6 | 87.3 | 76.8 | 42.3 |
| =15(μmol/L) | 102 | 139 | 110 | 736 | 48.1 | 84.1 | 42.3 | 84.1 |
| =20(μmol/L) | 68 | 173 | 28 | 818 | 70.8 | 82.5 | 28.2 | 96.7 |

We further assessed MTHFR 677TT genotype in predicting the risk of elevated Hcy level, compared to 677CC+CT genotype, using Logistic Regression analysis with adjustment for the pertinent covariates. As shown in TABLE 5, regardless of the cutoff points, MTHFR 677TT homozygosity is associated with higher risk of elevated plasma Hcy level, and MTHFR 677CC+CT genotype is associated with lower risk of elevated plasma Hcy level. Furthermore, the higher the cutoff point, the higher the risk of elevated plasma Hcy level as measured by odd ratio (OR) associated with MTHFR 677TT genotype compared to 677CC+CT genotype. Specifically, the OR is 5.7 (95% CI 2.1-8.6), 11.2 (95% CI 3.7-32.4), and 27.5 (95% CI 12.1-87.9) for the cutoff point of 10, 15, and 20 μmol/L, respectively. All of these results provide coherent evidence that MTHFR 677TT genotype can predict elevated plasma Hcy level, while the MTHFR 677CC+CT genotype predicts lower levels of plasma Hcy level.

TABLE 5
Association of MTHFR C677T polymorphism (TT vs. CC + CT)
with the risk of elevated plasma homocysteine (Hcy) level
defined by three different cutoff points.

| Cutoff point/ | | hHcy* | | | | |
|---|---|---|---|---|---|---|
| Genotype* | N | N | % | OR | 95% CI | P^{#} |
| =10(μmol/L) | | | | | | |
| CC + CT | 846 | 461 | 54.5 | 1.00 | — | — |
| TT | 241 | 185 | 76.8 | 5.7 | 2.1-8.6 | 0.00 |
| =15(μmol/L) | | | | | | |
| CC + CT | 846 | 110 | 13.0 | 1.00 | — | — |
| TT | 241 | 102 | 42.3 | 11.2 | 3.7-32.4 | 0.00 |
| =20(μmol/L) | | | | | | |
| CC + CT | 846 | 28 | 3.3 | 1.00 | — | — |
| TT | 241 | 68 | 28.2 | 27.5 | 12.1-87.9 | 0.00 |

^{#}Adjusted for sex, age, height, and weight
*Hyperhomocysteinemia

### Example 2

Determine Genotype of MTHFR Gene C677T Polymorphism (Ala222Val, dsSNP ID: rs1801133) and Predict the Risk for the Development and Prognosis of Cerebral and Cardiovascular Diseases.

2.1 Determine the Genotype of MTHFR C677T Polymorphism.

The Genotyping Method and Procedure is the Same as Described in Example 1.1.

2.2 Predict the Risks for the Development and Prognosis of Cerebral and Cardiovascular Diseases.

MTHFR 677TT genotype predicts a higher risk and a worse prognosis for cerebral and cardiovascular diseases.
MTHFR 677CC or 677CT genotypes predict a lower risk and a better prognosis for cerebral and cardiovascular diseases.
Hypertensive subjects with MTHFR 677TT genotype have higher risk and worse prognosis of cerebral and cardiovascular diseases.
Hypertensive subjects with MTHFR 677CC or 677CT genotypes have lower risk and better prognosis of cerebral and cardiovascular diseases.
MTHFR 677TT genotype and hyperhomocysteinemia predict a higher risk and worse prognosis of cerebral and cardiovascular diseases.
MTHFR 677CC or 677CT genotypes and hyperhomocysteinemia predict a lower risk and better prognosis of cerebral and cardiovascular diseases.
Hypertensive subjects with MTHFR 677TT genotype and hyperhomocysteinemia predict a higher risk and worse prognosis of cerebral and cardiovascular diseases.
Hypertensive subjects with MTHFR 677CC or 677CT genotype and hyperhomocysteinemia have a lower risk and better prognosis of cerebral and cardiovascular diseases.

All the above results were obtained from our prospective nested case-control study, in which a total of 39,165 eligible subjects were followed prospectively for a mean of 6.2 years. By the end of 2006, we have identified 204 new cases of cerebral and cardiovascular events. Of them, 148 cases had complete genotyping data on MTHFR C677T polymorphism. By one-to-one matching on sex, age, and community, we identified corresponding control subjects. We also obtained clinical data (including blood pressure, plasma Hcy level, blood glucose, serum lipids) and epidemiological data (including sex, age, height, weight, and BMI). We performed statistical analyses among three case-control groups: (1) all the cases with cerebral and cardiovascular events and matched controls (n=148 pairs); (2) all the cases with stroke and matched controls (n=133 pairs); and (3) all the cases of death associated with cerebral and cardiovascular diseases (n=102 pairs).

TABLE 6
Distribution of MTHFR C677T genotypes by the three case-control groups
(all cerebral and cardiovascular events; stroke, and death).

| | All cerebral cardiovascular events | | Stroke | | Death | |
|---|---|---|---|---|---|---|
| Genotype | Control | Case | Control | Case | Control | Case |
| N | 148 | 148 | 133 | 133 | 102 | 102 |
| | N (%) | N (%) | N (%) | N (%) | N (%) | N (%) |

MTHFR C677T

| | | | | | | |
|---|---|---|---|---|---|---|
| CC | 55(37.2) | 50(33.8) | 49(36.8) | 45(33.8) | 34(37.8) | 29(32.2) |
| CT | 76(51.4) | 72(48.6) | 68(51.1) | 65(48.9) | 46(51.1) | 43(47.8) |
| TT | 17(11.5) | 26(17.6) | 16(12.0) | 23(17.3) | 10(11.1) | 18(20.0) |

Recessive Model

| | | | | | | |
|---|---|---|---|---|---|---|
| CC + CT | 131(88.5) | 122(82.4) | 117(88.0) | 110(82.7) | 80(88.9) | 72(80.0) |
| TT | 17(11.5) | 26(17.6) | 16(12.0) | 23(17.3) | 10(11.1) | 18(20.0) |

There was no significant difference in distributions or proportions of age, sex, cigarette smoking or alcohol consumption between each case-control group (see Table 7). In the subsequent statistical analyses, we assessed independent and joint effects of MTHFR C677T polymorphism, hypertension, and plasma Hcy level in predicting the incident risk and prognosis of cerebral and cardiovascular diseases

Table 6 presents the distribution of MTHFR C677T genotypes in cases and controls by the three case-control groups (all cerebral and cardiovascular events; stroke, and death). Compared to matched controls, cases tend to have higher proportions of MTHFR 677TT genotype. The pattern is consistent for all the three case-control groups. In the recessive model, these differences reached statistical significance. Our data indicate that MTHFR C677T polymorphism was associated with incident risks of all cerebral and cardiovascular events; stroke, and death. Subjects with MTHFR 677TT genotype are associated with higher risks while subjects with MTHFR 677CC or 677CT genotypes are associated with lower risks of all cerebral cardiovascular diseases, stroke, and death.

We estimated the effect of plasma Hcy level on the incident risk of cerebral cardiovascular events. As shown in Table 7, compared with controls, a significantly higher Hcy level was observed in cases with all cerebral cardiovascular events compared to controls (Mean±SD: 18.1±13.0 vs. 14.9±6.8 μmol/L). Log-transformed Hcy level was also higher in the cases than in the controls (Mean±SD: 2.8±0.4 vs. 2.6±0.3 μmol/L). A similar pattern was observed for stroke (18.2±13.6 vs. 15.2±7.0), and for death (19.1±14.2 vs. 15.4±7.9). Our data suggest that higher levels of plasma Hcy may be an important risk factor of all cerebral cardiovascular events, stroke, and death from cerebral cardiovascular diseases.

We also assessed the effect of hypertension on the incident risk of cerebral cardiovascular events. As shown in Table 7, the proportions of hypertension in cases vs. controls, respectively, were 79.1% and 50.0% for all cerebral and cardiovascular events; 82.0% and 48.9% for stroke; and 74.4% and 47.8% for death. These significant differences in proportions indicate that hypertension may be an important risk factor for incident cerebral and cardiovascular events and its associated death.

TABLE 7
Major epidemiological and Clinical characteristics of subjects by the three case-control groups.

| | All cerebral cardiovascular | | | | | |
|---|---|---|---|---|---|---|
| | Events | | Stroke | | Death | |
| Variable | Control | Case | Control | Case | Control | Case |
| N | 148 | 148 | 133 | 133 | 102 | 102 |
| | Mean ± SD | Mean ± SD | Mean ± SD | Mean ± SD | Mean ± SD | Mean ± SD |
| Age, years | 56.3 ± 11.1 | 57.1 ± 10.9 | 56.7 ± 10.9 | 57.2 ± 10.5 | 57.5 ± 11.5 | 58.7 ± 11.1 |
| BMI | 20.7 ± 2.7 | 21.6 ± 3.0** | 20.6 ± 2.7 | 21.6 ± 3.0 | 20.5 ± 2.7 | 21.2 ± 2.8* |
| Hcy μmol/L | 14.9 ± 6.8 | 18.1 ± 13.0** | 15.2 ± 7.0 | 18.2 ± 13.6* | 15.4 ± 7.9 | 19.1 ± 14.2* |
| Log-Hcy, | 2.6 ± 0.3 | 2.8 ± 0.5** | 2.7 ± 0.3 | 2.8 ± 0.5* | 2.7 ± 0.4 | 2.8 ± 0.5** |
| mol/L | N (%) | N (%) | N (%) | N (%) | N (%) | N (%) |
| Male gender | 107(72.3) | 107(72.3) | 96(72.2) | 96(72.2) | 67(74.4) | 67(74.4) |
| Smoking | 78(52.7) | 75(50.7) | 71(53.4) | 68(51.1) | 47(52.2) | 44(48.9) |
| Alcohol use | 42(28.4) | 41(27.7) | 38(28.6) | 37(27.8) | 23(25.6) | 24(26.7) |
| Hypertension | 74(50.0) | 117(79.1)** | 65(48.9) | 109(82.0)** | 43(47.8) | 67(74.4)** |

**P < 0.01, *P < 0.05

We further evaluated plasma Hcy levels in relation to the risks of the above cerebral and cardiovascular events using Logistic Regression analysis with adjustment for pertinent covariates. As Table 8 shows, subjects with Hcy levels' greater than 10 but less than 20 μmol/L and subjects with Hcy levels at 20 μmol/L or above have significantly higher risks for all the cerebral and cardiovascular events, stroke, or associated death compared to those with Hcy level <10 μmol/L. Moreover, there is a dose-response relationship. A trend test further confirmed that elevated Hcy level was positively and linearly associated with the risks for all the above cerebral and cardiovascular events.

TABLE 8
Association of plasma homocysteine (Hcy) level with
the risk of cerebral and cardiovascular events.

| Hcy level | | | Adjusted* | | |
|---|---|---|---|---|---|
| (μmol/L) | Total (N) | Case (%) | OR | 95% CI | P |

All cerebral and cardiovascular events

| | | | | | |
|---|---|---|---|---|---|
| Hcy < 10 | 42 | 13(31.0) | 1.0 | — | — |
| 10 = Hcy < 20 | 282 | 141(50.0) | 2.2 | 1.0-4.6 | 0.039 |
| Hcy = 20 | 54 | 35(64.8) | 3.7 | 1.4-9.4 | 0.006 |
| Trend Test | — | — | 1.9 | 1.2-3.0 | 0.007 |

Stroke

| | | | | | |
|---|---|---|---|---|---|
| Hcy < 10 | 35 | 12(34.3) | 1.0 | — | — |
| 10 = Hcy < 20 | 248 | 122(49.2) | 1.9 | 0.9-4.1 | 0.094 |
| Hcy = 20 | 49 | 32(65.3) | 4.1 | 1.5-10.7 | 0.005 |
| Trend Test | — | — | 1.8 | 1.1-2.9 | 0.030 |

Death from cerebral and cardiovascular events

| | | | | | |
|---|---|---|---|---|---|
| Hcy < 10 | 33 | 7(21.2) | 1.0 | — | — |
| 10 = Hcy < 20 | 188 | 97(51.6) | 3.9 | 1.5-9.8 | 0.004 |
| Hcy = 20 | 47 | 30(63.8) | 5.7 | 1.9-17.5 | 0.002 |
| Trend Test | — | — | 2.2 | 1.3-3.8 | 0.004 |

Death from strokes

| | | | | | |
|---|---|---|---|---|---|
| Hcy < 10 | 26 | 6(23.1) | 1.0 | — | — |
| 10 = Hcy < 20 | 159 | 80(50.3) | 3.3 | 1.2-9.2 | 0.021 |
| Hcy = 20 | 43 | 28(65.1) | 5.1 | 1.5-17.2 | 0.010 |
| Trend Test | — | — | 2.1 | 1.2-3.7 | 0.014 |

*Logistic Regression model adjusted for age, sex, BMI, smoking, alcohol drinking, blood lipid, blood glucose, and blood pressure.

We evaluated joint effect of hyperhomocysteinemia and hypertension on the risks of cerebral and cardiovascular events and associated death. As Table 9 shows, after adjusting for age, sex, BMI, smoking, drinking, blood lipid and blood glucose, the odds ratios (ORs) for subjects with hypertension and hyperhomocysteinemia compared to the reference group were 9.5 (95% CI: 2.7-33.7) for all cerebral cardiovascular events, 12.1 (2.6-55.6) for stroke, 9.4 (2.6-34.4) for death from cerebral and cardiovascular event, and 11.8 (2.5-55.4) for death from stroke. Our data indicate that hypertension and hyperhomocysteinimia can jointly increase the risks for cerebral and cardiovascular events and associated death.

TABLE 9
Joint effect of hypertension and hyperhomocysteinemia
on the risk of cerebral and cardiovascular events

| Hyper- | | | | Adjusted# | | |
|---|---|---|---|---|---|---|
| tension* | hHcy** | Total (N) | Case (%) | OR | 95% CI | P |

All cerebral and cardiovascular events

| | | | | | | |
|---|---|---|---|---|---|---|
| No | No | 21 | 3(14.3) | 1.0 | — | — |
| No | Yes | 110 | 38(34.6) | 3.3 | 0.9-11.9 | 0.071 |
| Yes | No | 21 | 10(47.6) | 4.9 | 1.1-22.0 | 0.040 |
| Yes | Yes | 226 | 138(61.1) | 9.5 | 2.7-33.7 | <0.001 |

Stroke

| | | | | | | |
|---|---|---|---|---|---|---|
| No | No | 17 | 2(11.8) | 1.0 | — | — |
| No | Yes | 98 | 31(31.6) | 3.6 | 0.8-16.8 | 0.106 |
| Yes | No | 18 | 10(55.6) | 8.2 | 1.4-48.3 | 0.020 |
| Yes | Yes | 199 | 123(61.8) | 12.1 | 2.6-55.6 | 0.001 |

Death from all cerebral and cardiovascular events

| | | | | | | |
|---|---|---|---|---|---|---|
| No | No | 19 | 3(15.8) | 1.0 | — | — |
| No | Yes | 80 | 29(36.3) | 3.2 | 0.9-12.1 | 0.085 |
| Yes | No | 14 | 4(28.6) | 1.9 | 0.3-10.6 | 0.456 |
| Yes | Yes | 155 | 98(63.2) | 9.4 | 2.6-34.4 | <0.001 |

Death from stroke

| | | | | | | |
|---|---|---|---|---|---|---|
| No | No | 15 | 2(13.3) | 1.0 | — | — |
| No | Yes | 71 | 24(33.8) | 3.5 | 0.7-17.0 | 0.119 |
| Yes | No | 11 | 4(36.4) | 3.3 | 0.5-23.1 | 0.232 |
| Yes | Yes | 131 | 84(64.1) | 11.8 | 2.5-55.4 | 0.002 |

*Hypertension definition: SBP ≧ 140 mmHg or DBP ≧ 90 mmHg or taking antihypertensive drug.
**Hyperhomocysteinimia definition: Hcy ≧ 10 μmol/L.
#Adjusted for age, sex, BMI, smoking, drinking, blood lipid, and blood glucose.

TABLE 10
Logistic Regression for Estimating Joint Effect of MTHFR C677T Genetic Variant
and Hypertension on the risk of cerebral and cardiovascular events.

| Hyper | MTHFR | Total | Case | Case | Adjusted² | | | Adjusted³ | | |
|---|---|---|---|---|---|---|---|---|---|---|
| tension¹ | C677T⁴ | (N) | (N) | (%) | OR | 95% CI | P | OR | 95% CI | P |

All cerebral and cardiovascular events

| | | | | | | | | | | |
|---|---|---|---|---|---|---|---|---|---|---|
| No | 677CC or CT | 92 | 27 | 29.3 | 1.0 | | | 1.0 | | |
| No | 677TT | 13 | 4 | 30.8 | 0.9 | 0.3-3.5 | 0.938 | 0.7 | 0.2-2.9 | 0.622 |
| Yes | 677CC | 161 | 95 | 59.0 | 3.3 | 1.8-6.2 | <0.001 | 3.1 | 1.7-5.9 | <0.001 |
| | or CT | | | | | | | | | |
| Yes | 677TT | 30 | 22 | 73.3 | 6.4 | 2.3-17.6 | <0.001 | 4.6 | 1.5-13.8 | 0.007 |

Stroke

| | | | | | | | | | | |
|---|---|---|---|---|---|---|---|---|---|---|
| No | 677CC | 80 | 21 | 26.3 | 1.0 | | | 1.0 | | |
| | or CT | | | | | | | | | |
| No | 677TT | 12 | 3 | 25.0 | 0.8 | 0.2-3.6 | 0.820 | 0.7 | 0.2-3.4 | 0.702 |
| Yes | 677CC | 147 | 89 | 60.5 | 4.3 | 2.2-8.6 | <0.001 | 4.2 | 2.1-8.5 | <0.001 |
| | or CT | | | | | | | | | |
| Yes | 677TT | 27 | 20 | 74.1 | 8.2 | 2.7-24.7 | <0.001 | 7.0 | 2.1-23.5 | 0.002 |

Death from cerebral and cardiovascular events

| | | | | | | | | | | |
|---|---|---|---|---|---|---|---|---|---|---|
| No | 677CC | 69 | 22 | 31.9 | 1.0 | | | 1.0 | | |
| | or CT | | | | | | | | | |
| No | 677TT | 8 | 3 | 37.5 | 1.1 | 0.2-5.6 | 0.889 | 0.7 | 0.1-4.2 | 0.742 |
| Yes | 677CC | 103 | 60 | 58.3 | 2.8 | 1.4-5.8 | 0.005 | 2.6 | 1.2-5.3 | 0.012 |
| | or CT | | | | | | | | | |
| Yes | 677TT | 24 | 17 | 70.8 | 5.3 | 1.7-16.5 | 0.004 | 3.4 | 1.0-11.6 | 0.055 |

¹Hypertension definition: SBP >= 140 mmHg or DBP >= 90 mmHg, or taking antihypertensive drug.
²Adjusted for age, sex, BMI, smoking, drinking, blood lipid, and blood glucose.
³Adjusted for age, sex, BMI, smoking, drinking, blood lipid, blood glucose, and log-transformed Hcy level.

We further assessed the joint effect of hypertension and MTHFR C677T polymorphism on the risks of cerebral and cardiovascular events after adjusting for pertinent covariates. As shown in Table 10, the adjusted odds ratio (95% CI) for subjects with hypertension and MTHFR 677TT genotype compared to subjects without hypertension and with MTHFR 677CC or CT genotype was 6.4 (2.3 to 17.6) for all cerebral and cardiovascular events; 8.2 (2.7 to 24.7) for stroke; and 5.3 (1.7 to 16.5) for the associated death. By further inclusion of plasma Hcy level in the model, the adjusted odds ratio for subjects with hypertension and MTHFR 677TT genotype compared to subjects without hypertension and with MTHFR 677CC or CT genotype was 4.6 (1.5 to 13.8) for all cerebral and cardiovascular events, 7.0 (2.1 to 23.5) for stroke, and 3.4 (1.0 to 11.6) for associated death. Our data indicate that hypertensive subjects homozygous for the MTHFR 677T allele are at much greater risk for all cerebral and cardiovascular events, stroke, and associated death, compared to subjects without hypertension and with MTHFR 677CC or CT genotype.

We further limited the above analyses to subjects with baseline plasma Hcy level less than the 90th percentile of plasma Hcy level among corresponding control group. The adjusted odds ratio (95% CI) for subjects with hypertension and MTHFR 677TT genotype compared to subjects without hypertension and with MTHFR 677CC or CT genotype was 14.5 (2.8 to 75.3) for all cerebral and vascular events, 15.5 (2.8 to 86.0) for stroke, and 26.8 (3.0 to 236.3) for associated death. The adjusted odds ratios either remained the same or became even stronger after including plasma Hcy level into the model. Similarly, we performed the above analyses limited to subjects with baseline plasma Hcy level equal or less than 20 μmol/, and we observed similar results.

As demonstrated from the above data, there are consistent and significant joint associations of the MTHFR C677T polymorphism and hypertension with the risks of all cerebral and cardiovascular events, stroke, and associated death. Such associations were even more pronounced after excluding subjects with Hcy levels above 90^{th} percentile of Hcy level in corresponding control group or above 20 umol/L.

TABLE 11
Joint effect of hypertension and MTHFR C677T polymorphism on the risk of cerebral and
cardiovascular events among subjects with normal or moderately elevated Hcy level.

| | | | Case | Case | Adjusted² | | | Adjusted³ | | |
|---|---|---|---|---|---|---|---|---|---|---|
| Hypertension¹ | MTHFR C677T | N | (N) | (%) | OR | 95% CI | P | OR | 95% CI | P |

Limited to subjects with Hcy level less than 90^{th} percentile of Hcy level in
corresponding control group
All cerebral and cardiovascular events

| | | | | | | | | | | |
|---|---|---|---|---|---|---|---|---|---|---|
| No | 677CC or CT | 86 | 23 | 26.7 | 1.0 | | | 1.0 | | |
| No | 677TT | 9 | 2 | 22.2 | 0.8 | 0.1-4.3 | 0.795 | 0.9 | 0.2-5.2 | 0.935 |
| Yes | 677CC or CT | 142 | 80 | 56.3 | 3.3 | 1.7-6.5 | <0.001 | 3.5 | 1.8-7.1 | <0.001 |
| Yes | 677TT | 13 | 11 | 84.6 | 14.5 | 2.8-75.3 | 0.001 | 16.3 | 3.1-86.7 | 0.001 |

Stroke

| | | | | | | | | | | |
|---|---|---|---|---|---|---|---|---|---|---|
| No | 677CC or CT | 75 | 18 | 24.0 | 1.0 | | | 1.0 | | |
| No | 677TT | 9 | 2 | 22.2 | 0.9 | 0.2-5.1 | 0.928 | 1.2 | 0.2-6.9 | 0.839 |
| Yes | 677CC or CT | 130 | 76 | 58.5 | 4.6 | 2.1-9.7 | <0.001 | 5.2 | 2.4-11.4 | <0.001 |
| Yes | 677TT | 11 | 9 | 81.8 | 15.5 | 2.8-86.0 | 0.002 | 18.5 | 3.2-105.3 | 0.001 |

Death from cerebral and cardiovascular events

| | | | | | | | | | | |
|---|---|---|---|---|---|---|---|---|---|---|
| No | 677CC or CT | 65 | 19 | 29.2 | 1.0 | | | 1.0 | | |
| No | 677TT | 6 | 2 | 33.3 | 1.2 | 0.2-8.0 | 0.839 | 1.2 | 0.2-7.9 | 0.863 |
| Yes | 677CC or CT | 89 | 49 | 55.1 | 2.8 | 1.3-6.1 | 0.010 | 2.7 | 1.2-6.1 | 0.014 |
| Yes | 677TT | 11 | 10 | 90.9 | 26.8 | 3.0-236.3 | 0.003 | 25.6 | 2.8-234.2 | 0.004 |

Limited to subjects with Hcy level less than 20 umol/L
All cerebral and cardiovascular events

| | | | | | | | | | | |
|---|---|---|---|---|---|---|---|---|---|---|
| No | 677CC or CT | 87 | 23 | 26.4 | 1.0 | | | 1.0 | | |
| No | 677TT | 9 | 2 | 22.2 | 0.8 | 0.2-4.4 | 0.809 | 0.9 | 0.2-5.2 | 0.939 |
| Yes | 677CC or CT | 143 | 81 | 56.6 | 3.5 | 1.8-6.8 | <0.001 | 3.7 | 1.9-7.4 | <0.001 |
| Yes | 677TT | 13 | 11 | 84.6 | 15.0 | 2.9-77.7 | 0.001 | 16.7 | 3.2-88.3 | <0.001 |

Stroke

| | | | | | | | | | | |
|---|---|---|---|---|---|---|---|---|---|---|
| No | 677CC or CT | 76 | 18 | 23.7 | 1.0 | | | 1.0 | | |
| No | 677TT | 9 | 2 | 22.2 | 0.9 | 0.2-5.2 | 0.943 | 1.2 | 0.2-7.1 | 0.817 |
| Yes | 677CC or CT | 130 | 76 | 58.5 | 4.7 | 2.2-10.0 | <0.001 | 5.4 | 2.5-11.7 | <0.001 |
| Yes | 677TT | 11 | 9 | 81.8 | 16.0 | 2.9-88.6 | 0.002 | 19.1 | 3.4-108.9 | <0.001 |

Death from cerebral and cardiovascular events

| | | | | | | | | | | |
|---|---|---|---|---|---|---|---|---|---|---|
| No | 677CC or CT | 65 | 19 | 29.2 | 1.0 | | | 1.0 | | |
| No | 677TT | 6 | 2 | 33.3 | 1.3 | 0.2-8.4 | 0.797 | 1.3 | 0.2-8.8 | 0.785 |
| Yes | 677CC or CT | 87 | 47 | 54.0 | 2.6 | 1.2-5.6 | 0.017 | 2.6 | 1.2-5.8 | 0.019 |
| Yes | 677TT | 9 | 8 | 88.9 | 20.2 | 2.2-187.0 | 0.008 | 20.7 | 2.2-196.3 | 0.008 |

¹Hypertension definition: SBP >= 140 mmHg or DBP >= 90 mmHg, or taking antihypertensive drug
²Adjusted for age, sex, BMI, smoking, drinking, blood lipid, and blood glucose.
³Adjusted for age, sex, BMI, smoking, drinking, blood lipid, blood glucose, and log-transformed Hcy.

### Example 3

Determine Genotypes of MTHFR C677T (Ala222Val, dsSNP ID: rs1801133) Polymorphism and Predict the Treatment Effect of ACEI Medicine and ACEI Medicine Combined with B-Vitamin.

3.1 Determine the Genotype of MTHFR C677T Polymorphism.

The Method is the Same as Described in the Example 1.1 Section.

3.2 Predict the Treatment Effect of ACEI Medicine.

ACEI medicine treatment can cause an increased level of plasma Hcy in a subject. Moreover, such treatment induced increase in plasma Hcy level is greater among subjects with MTHFR 677TT genotype, but is less in subjects with MTHFR 677CC or 677CT genotypes.
For MTHFR 677TT carriers, combination therapy with ACEI medicine and B-vitamin is more effective in reducing plasma Hcy level, while for MTHFR 677CC or 677CT carriers the combination therapy is less effective in reducing plasma Hcy level.
For MTHFR 677TT carriers, combination therapy with ACEI and B-vitamin is more effective in reducing blood pressure level, while for MTHFR 677CC or 677CT carriers the combination therapy is less effective in reducing blood pressure level.
For MTHFR 677TT carriers, combination therapy with ACEI and B-vitamin is more effective in preventing damage to targeted organs such as kidney, while for MTHFR 677CC or 677CT carriers the combination therapy is less effective in preventing damage to targeted organs.

Here, ACEI medicine is specified as Enalapril, and B-vitamin is specified as folate.

We conducted a clinical epidemiology study, in which we classified hypertensive subjects into 677CC, 677CT, and 677TT groups based on the genotypes of MTHFR C677T polymorphism. Each subject was treated with Enalapril alone or combined with folate for 57 consecutive days. Pre-treatment and post-treatment plasma Hcy level, blood pressure (SBP/DBP), and urine albumin were measured. The major findings are summarized as follow.

Subjects treated with Enalapril alone for 57 days had an increased level of plasma Hcy. However, the degree of the increase in Hcy level was associated with the genotype of C677T polymorphism. Compared to subjects with 677CT or 677CC genotype, subjects with 677TT genotype had a significantly greater increase in plasma Hcy level post Enalapril treatment. Such finding persisted after adjustment for pertinent covariates. There was no significant difference in the increase of Hcy level between subjects with 677CT and subjects with 677CC genotype. These results suggest an association between MTHFR C677T polymorphism and ENALAPRIL treatment-induced increase in plasma Hcy level, which was particularly pronounced among subjects with MTHFR 677TT genotype.

TABLE 12a
The effect of MTHFR C677T polymorphism on the change
in plasma homocysteine (Hcy) level (umol/L) after
treatment with ENALAPRIL for 57 days.

| MTHFR | | Mean change* | | | |
|---|---|---|---|---|---|
| genotyp | N | in Hcy level | SD | β*(SE) | P |
| CC | 24 | −0.5 | 3.0 | 0 | |
| CT | 27 | 1.2 | 2.7 | 0.7 (1.3) | 0.582 |
| TT | 26 | 2.7 | 3.8 | 3.9 (1.3) | 0.002 |

*The multiple linear regression model adjusted for sex, age, BMI, smoking, drinking and baseline Hcy level.
# The change in plasma Hcy level is defined as the Hcy level at day 57^{th} minus Hcy level at the base line (Day 57 − Day 0).

As shown in Table 12b, we further combined subjects with MTHFR 677CC and 677CT genotype as a group, and defined significant change in Hcy level as (₅₇^{th} day's Hcy level-0^{th} day's Hcy level)/0^{th} day's Hcy level equal or greater than 10%; and non-significant change in Hcy level as (57^{th} day's Hcy level-0^{th} day's Hcy level)/0^{th} day's Hcy level less than 10%. Overall, 28.6% (22/77×100%) of the subjects had significant change in Hcy level. 38.5% (10/(10+16)×100%) of the subjects with the 677TT genotype had significant change in Hcy level. In contrast, only 23.3%(12(12+39)×100%) of the subjects with 677CT or CC had significant change in Hcy level. The data further indicate that MTHFR genotype affects Enalapril treatment-induced significant change in plasma Hcy level, and such treatment effect is more pronounced among subjects with 677TT genotype.

TABLE 12b
The Effect of MTHFR C677T Polymorphism on
significant change in plasma homocysteine
(Hcy) level post ENALAPRIL treatment.

| MTHFR | Significant* | Non-significant* | |
|---|---|---|---|
| genotype | change in Hcy | change in Hcy | Total |
| TT | 10 | 16 | 26 |
| CC + CT | 12 | 39 | 51 |
| Total | 22 | 55 | 77 |

*Significant change is defined as (57^{th} day's Hcy level-0^{th} day's Hcy level)/0^{th} day's Hcy level ≧10%; non-significant change is defined as (57^{th} day's Hcy level-0^{th} day's Hcy level)/0^{th} day's Hcy level <10‰

TABLE 13
Effect of MTHFR C677T polymorphism on Hcy-lowering response
induced by the combination therapy of ENALAPRIL and Folate.

| | | Mean Hcy-lowering | | | |
|---|---|---|---|---|---|
| Genotype | N | response # | SD | ? (SE) | P |
| CC | 43 | 0.69 | 3.52 | 0.0 | |
| CT | 52 | 2.15 | 3.91 | 2.8 (1.2) | 0.019 |
| TT | 49 | 3.44 | 4.90 | 4.1 (1.1) | 0.001 |

# the Hcy-lowering response is defined as 0^{th} day's Hcy level − 57^{th} day's Hcy level
* Adjusted for age, sex, BMI, smoking, drinking, and baseline Hcy level in plasma.

For hypertensive patients treated with a combination of ENALAPRIL and folate for 57 consecutive days, the MTHFR C677T polymorphism affects the Hcy-lowering response induced by the treatment. Here, the Hcy-lowering response is defined as 0^{th} day's Hcy level-57^{th} day's Hcy level. As shown in Table 13, the more 677T alleles carried by a subject, the greater the Hcy-lowering response post the treatment. Compared to subjects with 677CC genotype, subjects with 677TT genotype had a significantly greater plasma Hcy-lowering response. However, there was no significant difference between 677CT and 677CC groups. Our data indicate that the MTHFR C677T polymorphism can predict Hcy-lowering response induced by the combination therapy of ENALAPRIL and folate.

As shown in Table 14, among hypertensive subjects treated with a combination therapy of ENALAPRIL and folate for 57 consecutive days, the MTHFR C677T polymorphism affects its blood pressure-lowering response. The more 677T alleles carried by a subject, the greater the blood pressure-lowering response to the combination therapy. Compared to subjects with 677CC, subjects with 677TT had significantly greater blood pressure-lowing response, which is true for both systolic and diastolic blood pressure. However, there was no significant difference between the 677CT and CC groups. Our data indicate that the MTHFR C677T polymorphism can predict blood pressure-lowering response induced by the combination therapy, and such treatment response is more pronounced among subjects with the MTHFR 677TT genotype.

TABLE 14
Effect of the MTHFR C677T Polymorphism on blood
pressure-lowering response induced by the combination
therapy of ENALAPRIL and Folate.

| | | | | | |
|---|---|---|---|---|---|
| | | Mean BP | | | |
| MTHFR | | lowering | | | |
| Genotype | N | response# | SD | β(SE) | P |

Systolic blood pressure (mmHg)

| | | | | | |
|---|---|---|---|---|---|
| CC | 43 | 10.1 | 16.6 | 0 | |
| CT | 52 | 13.1 | 15.1 | 1.5 (2.8) | 0.581 |
| TT | 49 | 17.5 | 17.3 | 5.4 (2.6) | 0.032 |

Diastolic blood pressure (mmHg)

| | | | | | |
|---|---|---|---|---|---|
| CC | 43 | 4.2 | 6.3 | 0 | |
| CT | 52 | 5.1 | 7.5 | 0.4 (1.5) | 0.782 |
| TT | 49 | 7.3 | 7.0 | 2.6 (1.4) | 0.025 |

#the blood pressure-lowering response is defined as 0^{th} day's BP level − 57^{th} day's BP level.
* Adjusted for age, sex, BMI, smoking, drinking, and baseline blood pressure.

As shown in Table 15, among hypertensive subjects treated with combination therapy of ENALAPRIL and folate for 57 consecutive days, their urine albumin level was significantly reduced. Moreover, the MTHFR C677T polymorphism affects this urine albumin reduction effect, which is defined as 0^{th} day's urine albumin level-57^{th} day's urine albumin level. Compared to subjects with 677CC genotype, subjects with 677TT genotype had a significantly greater reduction in urine albumin level, but there is no significant difference between 677CT and 677 CC groups. Our data indicate that the MTHFR C677T polymorphism can predict urine albumin reduction effect induced by the combination therapy.

TABLE 15
The effect of MTHFR C677T polymorphism on urine
albumin reduction effect induced by the combination
therapy of ENALAPRIL and Folate.

| | | Mean urine | | | |
|---|---|---|---|---|---|
| MTHFR | | albumin reduction | | | |
| Genotype | N | (mg/24 hr)# | SD | β(SE) | P |
| CC | 43 | 13.1 | 5.8 | 0 | |
| CT | 52 | 15.7 | 6.1 | 1.1 (1.5) | 0.645 |
| TT | 49 | 20.4 | 4.9 | 6.8 (1.3) | <0.001 |

#the urine albumen reduction is defined as 0^{th} day's urine albumin level − 57^{th} day's urine albumin level.
* Adjusted for sex, age, BMI, cigarette smoking, drinking consumption, and baseline urine albumin.

In this case, ACEI medicine may also be BENALAPRIL, Lisinopril, or Fosinopril. B-vitamin refers to folate. (please verify?)

3.3 Predict Treatment Effect of Medicine Compounds Comprising ACEI Medicine Using a Subject's MTHFR Genotype and other Variables.

1. The data include MTHFR C677T polymorphism, age, sex, height, weight, history of smoking and drinking, occupation, education, baseline DBP, and SBP.

2. On the basis of multiple linear regression analysis, to construct multivariate model for predicting the treatment effects.

Prediction Equation of Blood Pressure-Lowering Response

(1) Prediction equation of DBP-lowering response:
? DBP=13.957+2.6017×677TT+0.8126×677CT−0.5771×age+0.1177×BMI+0.3632×gender−0.0847×history of drinking+0.30978×history of smoking−0.0416×height−0.0485×weight+0.4611×occupation+0.5295×education+0.2261×baseline DBP

? DBP is defined as baseline diastolic blood pressure minus 57^{th} day's diastolic blood pressure.

(2 ) Prediction equation of SBP-lowering response:
? SBP=13.099+5.3826×677TT+0.7826×677CT−0.7892×age+0.8902×BMI+0.2019×gender−0.8913×history of drinking+0.3892×history of smoking−0.0232×height−0.0312×weight+0.3145×occupation+0.4265×education+0.4917×baseline SBP

? SBP denoted as baseline systolic blood pressure minus 57^{th} day's systolic blood pressure

In the above equation, how to determine a genotyping value for a specific subject is described below: For a subject bearing a TT genotype, we defined the variable 677TT as “1”, and “0” otherwise; for a subject bearing a CT genotype, we defined the variable 677CT as “1”, and “0” otherwise; for a subject bearing a CC genotype, we defined the variables of 677TT and 677CT as “0”.

The covariates in the equation are defined as follow: age in years; BMI calculated as weight/height² (kg/m²); gender (“0” for male, “1” for female); history of alcohol consumption (“0” for never drinking, “1” for ever or current drinking); history of cigarette smoking (“0” for never smoking, “1” for ever or current smoking); height as the actual measurement with units of cm; weight as actual measurement with units of kg; occupation (“0” for farmer, “1” for non-farmer); education (“0” for middle or higher education, “1” for others); and baseline DBP and SBP as actual measurements with units of mmHg.

3. Using the above prediction equations, we can further quantify therapeutic efficacy of medicine compound comprising ACEI medicine.

According to the standard of cardiovascular medicine research guide entitled “The Clinical Criteria of Research and Development of New Medicine” issued by P. R. China, antihypertensive therapeutic response is defined as high efficacy, efficacy, or inefficacy.
1) High efficacy is defined as post-treatment DBP-lowering response ≧10 mmHg and its absolute DBP value in normal range(=90 mmHg); or DBP-lowering response ≧20 mmHg.
2) Efficacy is defined as post-treatment DBP-lowering response <10 mmHg but its absolute DBP value in normal range; or DBP-lowering response in range of 10 to 20 mmHg; or SBP-lowering response =30 mmHg.
3) Inefficacy: post-treatment BP-lowering response does not reach the standards for high efficacy or efficacy as described above.

In the analysis we combined high efficacy and efficiency into one group designated as “therapeutic efficacy”, otherwise as “therapy inefficacy”.

### Example 4

Determine MTHFR A1298C (Glu 429 Ala, dbSNP ID: rs1801131) Polymorphism and Predict Treatment Effect of Medicine Compound Comprising ACEI Medicine.

4.1 Determine MTHFR A1298C Polymorphism

1) Extract genomic DNA from blood sample according to the method described in Example 1.

2) To determine the genotype of MTHFR A1298C polymorphism by Taqman method.

a. Amplify the sequence including the MTHFR A1298C loci and flanking sequence by PCR thermocycle instrument. The 5 μl reaction system include 10 ng genomic DNA, 2.5 μl Taqman 2X Universal PCR Master Mix No AmpErase UNG (constituents include AmpliTaq Gold DNA Polymerase, dNTPs with dUTP, Passive reference, optimized buffer), 0.72 μM positive and negative primer each, 0.16 μM allele-specific TaqMan probes with fluorogenic reporter & quencher dyes each.

The sequences of primers are:

Positive primer: 5′ GGAGGAGCTGCTGAAGATGTG 3′
Negative primer: 5′ TGGTTCTCCCGAGAGGTAAAGA 3′

The sequences of allele-specific probes are:

VIC-5′ CCAGTGAAGAAAGTGTC 3′-NFQ

Which indicates “A” allele carrying VIC reporter dye.

FAM-5′ CAGTGAAGCAAGTGTC 3′-NFQ

Which indicates “C” allele carrying FAM reporter dye.

Reaction condition of PCR:

Predenaturation at 95° C. for 10 minutes; Denaturation at 92° C. for 15 seconds, Annealing and extension of primers at 60° C. for 60 seconds, 50 cycles.

b. Detect fluorescence signal on the 7900 quantitative PCR thermocycle instrument.

Detection and genotyping is carried out using “Allelic Discrimination” software by 7900 fluroscence quantitative PCR thermocycle instrument:

FAM means 1298CC homozygote;

VIC means 1298AA homozygote;

Both FAM and VIC mean 1298 heterozygote.

4.2 Predict Treatment Effect of Medicine Compound Comprising ACEI Medicine:

Treatment with ACEI medicine can cause a greater increase in plasma Hcy level in subjects with MTHFR 1298AA genotype, but such treatment effect is weaker in subjects with MTHFR 1298CC or 1298AC genotype.
For MTHFR 1298AA carriers, ACEI medicine combined with B-vitamin can more effectively reduce plasma Hcy level, but for MTHFR 1298CC or 1298AC carriers such treatment effect is weaker.
For MTHFR 1298AA carriers, ACEI medicine combined with B-vitamin can more effectively reduce blood pressure level, but for MTHFR 1298CC or 1298AC carriers such treatment effect is weaker.
For MTHFR 1298AA carriers, ACEI medicine combined with B-vitamin can more effectively protect targeted organs, while for MTHFR 1298CC or 1298AC carriers such treatment effect is weaker.

In this section, ACEI medicine was specified to be Enazepril, Benazepril, Lisinopril, or Fosinopril (please verify?), and B-vitamin was specified to be folate.

In our clinical epidemiology study, we classified hypertensive patients into 1298AA, 1298AC, and 1298CC groups based on the genotype of MTHFR A1298C polymorphism. Each subject was treated with ACEI medicine alone or combined with folate for 57 consecutive days. Pre-treatment and post-treatment plasma Hcy level, blood pressure (SBP/DBP), and urine albumin were measured. The results are summarized as follow:

As shown in Table 16, subjects treated with ENAZEPRIL alone for 57 days had a significantly increased level of plasma Hcy. Moreover, such treatment effect varies by the number of 1298A alleles. Compared to subjects with 1298CC genotype, subjects with 1298AA genotype had a significantly higher increase in plasma Hcy level post Enazepril treatment, but there is no significant difference between 1298CC and 1298AC groups. After adjustment for pertinent covariates, the findings persisted. Our data indicate that the MTHFR A1298C polymorphism affects ENAZEPRIL treatment-induced increase in plasma Hcy level, which is more pronounced among subjects with MTHFR 1298AA genotype.

TABLE 16
The effect of MTHFR A1298C polymorphism on
ENAZEPRIL treatment-induced change in plasma
homocysteine (Hcy) level (umol/L).

| | | Mean | | | |
|---|---|---|---|---|---|
| MTHFR | | change in | | | |
| genotype | N | Hcy level# | SD | β(SE)* | P |
| CC | 8 | 1.1 | 1.7 | 0 | |
| AC | 51 | 1.2 | 1.9 | 0.2 (1.5) | 0.982 |
| AA | 141 | 3.0 | 3.2 | 2.3 (1.8) | 0.027 |

#The change in plasma Hcy is defined as the Hcy level on 57^{th} day minus Hcy level at the base line (Day 57 − Day 0).
*Adjusted for sex, age, BMI, smoking, drinking and baseline Hcy level.

As shown in Table 17, among hypertensive subjects treated with ENAZEPRIL combined with folate for 57 consecutive days, the MTHFR A1298C polymorphism affects the treatment induced-Hcy-lowering response. Moreover, such treatment response varied by the number of 1298A allele in a subject. Compared to subjects with 1298CC genotype, subjects with 1298AA genotype had a significantly greater Hcy-lowering response, but no significant difference was observed between 1298AC and 1298CC groups. Our data indicate that the MTHFR A1298C polymorphism can predict Hcy-lowering response induced by the treatment of ENAZEPRIL combined with folate, and such response is more pronounced among subjects with 1298AA genotype.

As shown in Table 17, among hypertensive subjects treated with ENAZEPRIL combined with folate for 57 consecutive days, their urine albumin level was significantly reduced. Moreover, this treatment induced-urine albubin reduction effect varied by the MTHFR A1298C polymorphism. Compared to subjects with 1298CC genotype, subjects with 1298AA had a more significant reduction of urine albumen level, but no difference between 1298AC and 1298CC groups was observed. Our data indicated that the MTHFR A1298C polymorphism can predict urine albumin reduction response induced by the treatment of ENAZEPRIL combined with folate, and such response is more pronounced among subjects with 1298AA genotype.

TABLE 17
Effect* of MTHFR A1298C polymorphism on Hcy-lowering
response and urine albumin-reduction response induced
by the treatment of ENAZEPRIL combined with Folate.

| | | | | | | Urine albumin- | | | |
|---|---|---|---|---|---|---|---|---|---|
| MTHFRG | | Hcy-lowering response# | | | | reduction response# | | | |
| eno- | | (umol/L, Day 0 − Day 57) | | | | (mg/24 hrs, Day 0 − Day 57) | | | |
| Type | N | Mean | SD | β(SE) | P | Mean | SD | β(SE) | P |
| CC | 8 | 1.5 | 3.0 | 0.0 | | 11.5 | 5.4 | 0 | |
| AC | 51 | 1.3 | 2.8 | −0.1 (1.1) | 0.962 | 13.1 | 7.3 | 1.3 (2.5) | 0.785 |
| AA | 141 | 4.2 | 3.7 | 3.1 (1.3) | 0.009 | 20.3 | 9.2 | 7.7 (2.3) | <0.001 |

*Adjusted for sex, age, BMI, cigarette smoking, drinking consumption.
#The Hcy-lowering response is defined as the Hcy level on day 0 minus Hcy level on day 57 (Day 0 − Day 57).
#The urine albumin-reduction response is defined as the urine albumin level on day 0 minus urine albumin level on day 57 (Day 0 − Day 57).

As shown in Table 18, among hypertensive subjects treated with ENAZEPRIL combined with folate for 57 consecutive days, MTHFR A1298C polymorphism affects treatment induced-blood pressure-lowering response. Moreover, such treatment response varied by the number of 1298A allele in a subject. Compared to subjects with 1298CC genotype, subjects with 1298AA genotype had a significantly greater blood pressure-lowing response, but no significant difference between 1298AC and 1298CC groups was observed. Our data indicate that the MTHFR A1298C polymorphism can predict blood pressure-lowering response induced by the treatment of ENAZEPRIL combined with folate, and such treatment response is more pronounced among subjects with 1298AA genotype.

TABLE 18
Effect* of MTHFR A1298C Polymorphism on blood
pressure-lowering response induced by the treatment
of ENAZEPRIL combined with folate.

| | | | | | |
|---|---|---|---|---|---|
| | | Mean BP | | | |
| MTHFR | | lowering | | | |
| Genotype | N | response | SD | β(SE) | P |

Mean SBP lowering response# (mmHg)

| | | | | | |
|---|---|---|---|---|---|
| CC | 8 | 12.3 | 12.7 | 0 | |
| AC | 51 | 14.4 | 12.4 | 1.1 (2.9) | 0.921 |
| AA | 141 | 25.1 | 12.1 | 8.5 (3.3) | 0.041 |

Mean DBP lowering response# (mmHg)

| | | | | | |
|---|---|---|---|---|---|
| CC | 8 | 4.7 | 6.4 | 0 | |
| AC | 51 | 5.2 | 5.9 | 0.5 (1.7) | 0.851 |
| AA | 141 | 7.5 | 4.4 | 2.3 (1.6) | 0.023 |

*Adjusted for sex, age, BMI, cigarette smoking, drinking consumption.
#The blood pressure-lowering response is defined as the SBP or DBP level on day 0 minus SBP or DBP level on day 57 (Day 0 − Day 57).

### Example 5

A Kit for Determining the Genotype of the MTHFR C677T Polymorphism and Predicting Plasma Hcy Level, Incident Risk and Prognosis of Cerebral and Cardiovascular Events.

5.1 Main components of kit: DNA extraction reagent, PCR reaction solution, MTHFR C677T specific primers, nuclear acid polymerase, restriction endonuclease, nuclease buffer, negative control, and positive control. The positive control includes a positive template with MTHFR C677T wild homozygote, mutant homozygote, and heterozygote genotype. Specific primers refer to those that can specifically amplify the sequence containing the MTFHR C677T polymorphism loci.

5.2 Procedure of DNA Extraction and Genotyping:

5.2.1 Extracting Genomic DNA from Blood Sample:

a Add 400 μl RBC lysis solution to 1.5 ml EP, then add 100 ul fresh or anticoagulated whole blood sample, mix gently, incubate the sample at 37° C. for 5 minutes. Centrifuge the sample for 1 minute at 15000×g.
b Discard the supernatant and add 100 ul white blood cell lysis solution, votexing for 30 seconds, incubation for 5 minutes at 37° C. Add 35 ul protein precipitation solution, votex for 20 seconds and centrifuge at 15000×g for 90 seconds. Brown pellets can be seen at the bottom.
c Transfer supernatant to 1.5 ml tube with 100 ul isopropyl alcohol and gently shake several times until forming white flocculation precipitation.
d Discard the supernatant and add 100 ul 75% ethanol (prepared with anhydrous alcohol) to wash the precipitation, centrifuge at 15000×g for 90 seconds, discard the supernatant and air dry at room temperature (what is RT??).
e Add 100 ul nuclear storage solution then obtain genomic DNA from whole blood.
f Detect the DNA concentration by ultraviolet spectrophotometry method. The DNA concentration (μg DNA/ml) can be calculated using the formula: (A260 reading)×(50 μg/ml constant)×(Dilution Factor), the DNA quality was evaluated by the 260/280 ratio, which is ideally at 1.8˜2.0.
   5.2.2 Detect MTHFR C677T Polymorphism Site by Polymerase Chain Reaction—Restriction Fragment Length Polymorphism (PCR-RFLP):

Design specific primers for PCR by MTHFR C677T gene sequence including forward primer and backward primer. PCR amplification is performed by routine method as described below.

The primer sequence:

Forward primer: 5′- CTT TGA GGC TGA CCT GAA GC-3′
Backward primer: 5′- CTG GGA AGA ACT CAG CGA AC-3′

Reaction system of PCR:
45 ng Genomic DNA, 10 pmol (20 μmol/L) of each of the two primers, 2.0 mmol/L dNTPs, 1.0 μl 10 × buffer, 3 U Gold Taq DNA polymerase, add ddH2O to 10 μl final volume.
   Reaction condition of PCR:
Predenaturation at 95° C. for 10 minutes; Denaturation at 94° C. for 30 seconds, Annealing of primers at 59° C. for 45 seconds, Extension of primers at 68° C. for 45 seconds, 35 cycles, extension at 68° C. for 7 minutes, and finally to acquire the 274 bp fragment.
   The condition and system of RFLP:
The reaction volume is 15 μl including 10 μl PCR product, 1.5 μl 10× NEBuffer#2, 4 U Hinf I endonuclease and 3.1 μl ddH2O. Incubation at 37° C. overnight.

The recognized site of Hinf I endonuclease is:

5′ . . . G [symbol] A N T C. . . 3′
3′ . . . C T N A [symbol] G. . . 5′

5.3 Determination of MTHFR Genotype:

The PCR products after endonuclease reaction are separated by electrophoresis and read under ultraviolet light. The MTHFR genotype is determined as follow:
If the restriction fragment length is 274 bp, the genotype is 677CC;
If the restriction fragment length are 274 bp and 228 bp and 46 bp, the genotype is 677CT;
If the restriction fragment length is 228 bp and 46 bp, the genotype is 677TT.
   5.4 Prediction of Plasma Hcy Level

We can predict plasma Hcy level based on the genotyping results obtained in the example 5.3 section, and the predictive approach described in the example 1.2 section.
MTHFR 677TT carriers tend to have a higher level of plasma Hcy, and a greater probability of increasing plasma Hcy level.
MTHFR 677CC or 677CT carriers tend to have a lower level of plasma Hcy, and a lower probability of increasing plasma Hcy level.
   (note, I deleted the statement on hcy assocated diseases)
   5.5 Prediction of Incident Risk and Prognosis of Cerebral and Cardiovascular Events

We can predict incident risk and prognosis of cerebral and cardiovascular events based on the genotyping results obtained in the Example 5.3 section, and the predictive approach described in the Example 2.2 section.
MTHFR 677TT genotype predicts a higher risk and a worse prognosis for cerebral and cardiovascular diseases.
MTHFR 677CC or 677CT genotypes predict a lower risk and a better prognosis for cerebral and cardiovascular diseases.
Hypertensive subjects with MTHFR 677TT genotype have higher risk and worse prognosis of cerebral and cardiovascular diseases.
Hypertensive subjects with MTHFR 677CC or 677CT genotypes have lower risk and better prognosis of cerebral and cardiovascular diseases.
MTHFR 677TT genotype and hyperhomocysteinemia predict a higher risk and worse prognosis of cerebral and cardiovascular diseases.
MTHFR 677CC or 677CT genotypes and hyperhomocysteinemia predict a lower risk and better prognosis of cerebral and cardiovascular diseases.
Hypertensive subjects with MTHFR 677TT genotype and hyperhomocysteinemia predict a higher risk and worse prognosis of cerebral and cardiovascular diseases.
Hypertensive subjects with MTHFR 677CC or 677CT genotype and hyperhomocysteinemia have a lower risk and better prognosis of cerebral and cardiovascular diseases.

### Example 6

A Kit for Determining Genotype of MTHFR C677T Polymorphism and Predicting Treatment Effect of Medicine Compounds Comprising ACEI Medicine.

6.1 The Main Components of the Kit are the Same as Described in Example 5.1 Section.

6.2 Procedure of Detecting MTHFR C677T Polymorphism Loci is the Same as Described in Example 5.2 Section.

6.3 Determination of MTHFR C677T Polymorphism Genotype is the Same as Described in Example 5.2 Section.

6.4 Prediction of Treatment Effects Induced by Medicine Compounds Comprising ACEI Medicine:

We can predict the treatment effects induced by the medicine compounds comprising ACEI medicine, based on the genotyping result of MTHFR C677T polymorphism obtained from the Example of 6.3 section and the same prediction approach described in the Example 3.2 section. (note, I added the following statements)
ACEI medicine treatment can cause an increased level of plasma Hcy in a subject. Moreover, such treatment induced increase in plasma Hcy level is greater among subjects with MTHFR 677TT genotype, but is less in subjects with MTHFR 677CC or 677CT genotypes.
For MTHFR 677TT carriers, combination therapy with ACEI medicine and B-vitamin is more effective in reducing plasma Hcy level, while for MTHFR 677CC or 677CT carriers the combination therapy is less effective in reducing plasma Hcy level.
For MTHFR 677TT carriers, combination therapy with ACEI and B-vitamin is more effective in reducing blood pressure level, while for MTHFR 677CC or 677CT carriers the combination therapy is less effective in reducing blood pressure level.
For MTHFR 677TT carriers, combination therapy with ACEI and B-vitamin is more effective in preventing damage to targeted organs such as kidney, while for MTHFR 677CC or 677CT carriers the combination therapy is less effective in preventing damage to targeted organs

### Example 7

A Kit for Determining Genotype of MTHFR A1298C Polymorphism and Predicting Treatment Effects of Medicine Compounds Comprising ACEI Medicine.

7.1 The Main Components of Kit:

DNA extraction reagent, PCR reaction solution, MTHFR A1298C genotype specific primers, specific probes, nuclear acid polymerase, negative control, positive control. The positive control includes positive template with MTHFR A1298C wild homozygote, mutant homozygote, and heterozygote genotype. The specific primers mean primers which can specifically amplify sequence including MTFHR A1298C polymorphism site, and specific probes mean probes which can specifically hybridize with wild type or mutant type of MTHFR A1298C polymorphism site. The preferable probe is Taqman probe.

7.2 Determination of the MTHFR A1298C Polymorphism Genotype:

7.2.1 The Procedure of DNA Extraction and Determination of MTHFR A1298C Polymorphism Site Follow the Procedures Described in the Example 1.1.

7.2.2 Determination of the MTHFR A1298C Genotype Uses the Taqman Method as Described in the Example 4.1 Section.

7.3 Predicting the Treatment Effect of Medicine Compounds Comprising ACEI Medicine:

We can predict the treatment effect of medicine compounds comprising ACEI medicine based on the genotyping result of MTHFR A1298C polymorphism obtained from the Example 7.2.2 section and the same prediction method as described in the Example 4.2 section. (I added the following statement)
Treatment with ACEI medicine can cause a greater increase in plasma Hcy level in subjects with MTHFR 1298AA genotype, but such treatment effect is weaker in subjects with MTHFR 1298CC or 1298AC genotype.
For MTHFR 1298AA carriers, ACEI medicine combined with B-vitamin can more effectively reduce plasma Hcy level, but for MTHFR 1298CC or 1298AC carriers such treatment effect is weaker.
For MTHFR 1298AA carriers, ACEI medicine combined with B-vitamin can more effectively reduce blood pressure level, but for MTHFR 1298CC or 1298AC carriers such treatment effect is weaker.
For MTHFR 1298AA carriers, ACEI medicine combined with B-vitamin can more effectively protect targeted organs such as kidney, while for MTHFR 1298CC or 1298AC carriers such treatment effect is weaker.

### Example 8

Determine the Genotype of MTHFR C677T Polymorphism (Ala222Val, dsSNP ID: rs1801133) and Predict the Treatment Effect of Medicine Compounds Comprising ACEI Medicine on Liver Function.

8.1 Determination of the MTHFR C677T Genotype Uses the Method Described in the Example 1.1 Section.

8.2 Prediction of the Treatment Effect

ACEI medicine can cause liver injury, which can be assessed by the elevated liver enzymes (ALT, AST). The MTHFR C677T polymorphism can affect the degree of liver injury by ACEI medicine with and without combination with folate. In this case, ACEI-medicine was specified to be ENAZEPRIL, B-vitamin was specified to be folate. The alternative could be BENAZEPRIL, Lisinopril, or Fosinopril.
As shown in Table 19 and 20, the MTHFR C677T polymorphism can affect the degree of liver injury induced by ACEI medicine. Subjects with MTHFR 677CC genotype tend to have greater increase in liver enzymes (ALT and AST), but subjects with MTHFR 677TT genotype tend to have less increase in liver enzymes (ALT and AST) induced by Enazepril treatment. Subjects with MTHFR 677CT genotype fell in-between.
As shown in Table 21, the MTHFR C677T polymorphism can affect the degree of liver injury induced by ACEI medicine combined with folate. Controlling for MTHFR C677T genotype, subjects treated with combination of Enazepril and folate had a smaller increase in ALT and AST. Remarkably, given the combination therapy, Subjects with MTHFR 677CC genotype tend to have greater increase in ALT and AST, but subjects with MTHFR 677TT genotype tend to have less increase in ALT and AST. Subjects with MTHFR 677CT genotype fell in-between. Our data indicate that the MTHFR C677T polymorphism and combination of Enazepril with folate can jointly affect the degree of liver injury, in which subjects with MTHFR 677TT genotype and treated with combination of Enazepril with folate showed no elevation in ALT and AST but subjects with MTHFR 677CC genotype and treated with Enazepril alone showed the greatest increase in ALT and AST.

In our clinical epidemiology study, we classified subjects into 677CC, 677CT, and 677TT groups based on the MTHFR C677T genotype. Each subject was treated with ENAZEPRIL alone or combined with folate for 57 consecutive days. Pre-treatment and post-treatment liver enzymes (ALT and AST) were measured. The results were described as below:

As shown in Table 19, the MTHFR C677T polymorphism can affect the degree of liver injury induced by Enazepril treatment. Subjects with MTHFR 677CC genotype tend to have greater increase in liver enzymes (ALT and AST), but subjects with MTHFR 677TT genotype tend to have less increase in liver enzymes (ALT and AST). Subjects with MTHFR 677CT genotype fell in-between.

TABLE 19
Effect* of MTHFR C677T Polymorphism on ENAZEPRIL-induced
liver injury as measured by elevated liver
enzymes (ALT and AST).

| | | | | |
|---|---|---|---|---|
| | | Mean change in | | |
| MTHFR Genotype | N | liver enzyme# | SD | P |

Mean change in ALT (U/L)

| | | | | |
|---|---|---|---|---|
| CC | 54 | 13.8 | 12.3 | |
| CT | 50 | 10.1 | 11.1 | 0.362 |
| TT | 46 | 7.3 | 6.2 | 0.004 |

Mean change in AST (U/L)

| | | | | |
|---|---|---|---|---|
| CC | 54 | 7.1 | 6.8 | |
| CT | 50 | 5.2 | 5.9 | 0.519 |
| TT | 46 | 0.3 | 2.1 | 0.001 |

*Adjusted for sex, age, BMI, cigarette smoking, and drinking consumption.
#The change in liver enzyme is defined as ALT or AST level on day 57 minus ALT or AST level on day 0.

As shown in Table 20, the MTHFR C677T polymorphism can affect the degree of change in ALT and AST induced by Enazepril combined with folate. Controlling for MTHFR C677T genotype, subjects treated with combination of Enazepril with folate had a smaller increase in ALT and AST than subjects treated with Enazepril alone. Remarkably, among subjects treated with the combination of Enazepirl and folate, subjects with MTHFR 677CC genotype tend to have greater increase in ALT and AST, but subjects with MTHFR 677TT genotype tend to have less increase in ALT and AST. Subjects with MTHFR 677CT genotype fell in-between. Our data indicate that the MTHFR C677T polymorphism and combination of Enazepril with folate can jointly affect the degree of liver injury, in which subjects with MTHFR 677TT genotype and the combination therapy showed no elevation in ALT and AST, but subjects with MTHFR 677CC genotype and treated with Enazepril alone showed the greatest increase in ALT and AST. The findings persisted after adjusting for sex, age, BMI, cigarette smoking, and drinking consumption.

TABLE 20
Joint effect of MTHFR C677T polymorphism and combination therapy
of ENAZEPRIL with Folate on the change in ALT and AST.

| | | | | | | |
|---|---|---|---|---|---|---|
| | | | Mean | | | |
| | | | chang in | | | |
| MTHFR | | Type of | liver | | | |
| Genotype | N | Treatment | enzyme | SD | T | P |

Mean change in ALT (U/L, Day 57 − Day 0)

| | | | | | | |
|---|---|---|---|---|---|---|
| CC | 52 | ENAZEPRIL | 17.92 | 12.3 | | |
| | 52 | ENAZEPRIL + FOLATE | 6.71 | 6.32 | 2.06 | 0.03 |
| CT | 52 | ENAZEPRIL | 10.15 | 9.1 | | |
| | 52 | ENAZEPRIL + FOLATE | 4.32 | 4.11 | 1.07 | 0.28 |
| TT | 48 | ENAZEPRIL | 9.4 | 6.2 | | |
| | 48 | ENAZEPRIL + FOLATE | −0.50 | 2.94 | 1.90 | 0.04 |

Mean change in AST (U/L, Day57 − Day0)

| | | | | | | |
|---|---|---|---|---|---|---|
| CC | 52 | ENAZEPRIL | 4.95 | 4.8 | | |
| | 52 | ENAZEPRIL + FOLATE | 1.93 | 2.0 | 0.7 | 0.05 |
| CT | 52 | ENAZEPRIL | 5.81 | 5.7 | | |
| | 52 | ENAZEPRIL + FOLATE | −0.84 | 1.97 | 1.44 | 0.06 |
| TT | 48 | ENAZEPRIL | 0.08 | 1.1 | | |
| | 48 | ENAZEPRIL + FOLATE | −2.14 | 1.1 | 0.73 | 0.04 |

TABLE 21
Effect of the MTHFR C677T Polymorphism on ENAZEPRIL-induced
significant change in ALT.

| MTHFR | Significant change | Insignificant change | |
|---|---|---|---|
| genotype | in ALT | in ALT | Total |
| CC | 32 | 20 | 52 |
| TT | 14 | 34 | 48 |
| Total | 46 | 54 | 100 |

Sensitivity: 32/46 × 100% = 69.6% Specificity: 34/54 × 100% = 59.3%
PPV: 32/52 × 100% = 61.5% NPV: 34/48 × 100% = 70.8%
*?² = 2.88, p = 0.09

We further assessed change in ALT and AST as a dichotomous variable, that is, to define significant change in liver enzyme as (ALT day 57-ALT day 0)/ALT day 0>15%. Otherwise, as insignificant change in liver enzyme. As Table 21 shows, the proportion of significant change in ALT is greater in subjects with 677CC genotype and lower in subjects with 677TT genotype.

### Example 9

A Kit for Predicting Blood Pressure-Lowering Response of ACEI Medicine in Hypertensive Subjects.

We applied our kit in a small-scale study of hypertensive patients treated with ACEI medicine. Available data include MTHFR C677T genotype, age, gender, height, weight, history of smoking and drinking, occupation, education, and baseline blood pressure. Based on the predictive method described in the Example of 3.3 section, we obtained predictive values for the treatment response of SBP and DBP, which was then classified as efficacy vs. inefficacy groups.

TABLE 22
Effect of MTHFR C677T polymorphism on blood pressure-lowering
response induced by the treatment of ACEI medicine.

| | Efficacy | Inefficacy | Total |
|---|---|---|---|
| | (actually observed) | (actually observed) | N |
| Efficacy (predicted) | 54 | 28 | 82 |
| Inefficacy (predicted) | 12 | 50 | 62 |
| Total N | 66 | 78 | 144 |

Sensitivity: 54/66 × 100% = 81.8%; Specificity: 50/78 × 100% = 64.1%
PPV: 54/82 × 100% = 65.9%; NPV: 50/62 × 100% = 80.6%

As shown in TABLE 22, we constructed a 2×2 table to evaluate the performance of the kit by comparing the consistency between the observed and the predicted treatment efficacy. Our kit demonstrated a sensitivity of 81.8%, specificity of 64.1%, PPV of 65.9%, and NPV of 80.6%. We anticipate that this kit will contribute to the clinical setting as a valuable screening tool in identifying hypertensive subjects who will benefit the most from ACEI medication treatment and its combination with folate.

What is claimed is:

1. A usage of methylenetetrahydrofolate reductase (MTHFR) gene polymorphisms in predicting homocysteine (Hcy) level and/or the incidence and prognosis of Hcy-associated diseases in a subject.

2. The usage of claim 1, wherein said MTHFR gene polymorphisms include at least C677T single nucleotide polymorphisms (SNP).

3. The usage of claim 1, wherein said MTHFR gene polymorphisms also include the SNPs selected from A1298C, G1793A, G215A, G482A, and A1317G, and any other polymorphisms that are in linkage disequilibrium with the above SNPs.

4. The usage of claim 1, wherein said Hcy-associated diseases include but not limited to atherosclerosis, coronary heart disease, cerebrovascular disease, impaired renal function, peripheral vascular disease, arteriovenous thrombosis disease, hypertension, dyslipidemia (abnormal serum lipids), diabetes, psychosis, acute cardio-cerebrovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, diaphragmatic angina, acute myocardial infarction, cardiac shock, and cardiogenic sudden death, in which stroke is the preferred disease.

5. The usage of claim 1, wherein
(1) the TT genotype of MTHFR gene C677T polymorphism predicts an increased level of Hcy and an increased risk for hyperHcymia (high Hcy level);
(2) the CC genotype of MTHFR gene C677T polymorphism predicts a decreased level of Hcy and a decreased risk for hyperHcymia (high Hcy level);
(3) the TT genotype of MTHFR gene C677T polymorphism predicts an increased risk of developing Hcy-associated diseases and poorer prognosis of theses diseases;
(4) the CC and/or CT genotypes of MTHFR gene C677T polymorphism predicts an decreased risk of developing Hcy-associated diseases and better prognosis of theses diseases.

6. The usage of claim 5, wherein said subject refers to the subject with hypertension or with hyperhomocystinemia.

7. The usage of claim 5, wherein said subject refers to the subject with hypertension and with hyperhomocystinemia.

8. A oligonucleotide fragment of determining the genotypes of MTHFR gene polymorphisms, said oligonucleotide fragment being the gene specific primer or allele specific oligonucleotide probe, said MTHFR gene polymorphisms at least including the C677T polymorphism, said oligonucleotide fragment is 15-50 bases long preferably.

9. The oligonucleotide fragment of claim 8, wherein said MTHFR gene polymorphisms also include the SNPs selected from A1298C, G 1793A, G215A, G482A, and A1317G and any other polymorphisms that are in linkage disequilibrium with the above SNPs.

10. A method of predicting Hcy level in biological specimens and the incidence and prognosis of Hcy-associated diseases by the usage of MTHFR gene polymorphisms in a subject, said selected SNPs of MTHFR gene including the C677T SNP at least, said method comprising the steps of: (a) determining genotypes of MTHFR gene polymorphisms by the oligonucleotide fragment of claim 8 or claim 9; (b) predicting Hcy level and the incidence and prognosis of Hcy-associated diseases by the usage of genotypes of the MTHFR gene polymorphisms.

11. The method of claim 10, wherein said MTHFR gene polymorphisms include the SNPs selected from A1298C, G1793A, G215A, G482A, and A1317G and any other polymorphisms that are in linkage disequilibrium with the above SNPs.

12. The method of claim 10, wherein said Hcy-associated diseases include but not limited to atherosclerosis, coronary heart disease, cerebrovascular disease, impaired renal function, peripheral vascular disease, Arteriovenous thrombosis disease, hypertension, hyperlipidemia, diabetes, psychosis, acute cardio-cerebrovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, diaphragmatic angina, acute myocardial infarction, cardiac shock, and cardiogenic sudden death, of which stroke is a preferred disease.

13. The method of claim 10, wherein
(1) the TT genotype of MTHFR gene C677T polymorphism predicts an increased level of Hcy and an increased risk for hyperHcymia (high Hcy level);
(2) the CC genotype of MTHFR gene C677T polymorphism predicts a decreased level of Hcy and a decreased risk for hyperHcymia (high Hcy level);
(3) the TT genotype of MTHFR gene C677T polymorphism predicts an increased risk of developing Hcy-associated diseases and poorer prognosis of theses diseases;
(4) the CC and/or CT genotypes of MTHFR gene C677T polymorphism predicts an decreased risk of developing Hcy-associated diseases and better prognosis of theses diseases.

14. The method of claim 13, wherein said subject refers to the subject with hypertension or with hyperhomocystinemia.

15. The method of claim 14, wherein said subject refer to the subject with hypertension and with hyperhomocystinemia.

16. The method of claim 10, wherein said method may be derived from all kinds of nucleic acid analytical techniques: polymerase chain reaction(PCR), polymerase chain reaction-restriction fragment length polymorphism(PCR-RFLP), PCR-allele specificity oligonucleotide probe (PCR-ASO), PCR-sequence specificity oligonucleotide (PCR-SSO), sequencing, PCR-sequence specificity primer (PCR-SSP), PCR-fluorometric method, PCR-finger-printing method, oligonucleotide ligation analysis, fluorescence energy resonance transfer detection, biochip, nucleic acid-chip, DNA-chip, mass spectrum, gene-scan, single strand conformation polymorphism (SSCP), denaturing gel gradient electrophoresis, enzyme or chemistry mismatch cutting method and Taqman method.

17. The method of claim 10, wherein said biological specimens include blood sample, body fluid sample, tissue sample, organ sample, and cultured cells, of which blood sample is the preferred biological specimen.

18. A kit of predicting Hcy level in biological specimen and/or the incidence and prognosis of Hcy-associated diseases in a subject using MTHFR gene polymorphisms: said selected SNPs of MTHFR gene including the C677T polymorphism at least; said Hcy-associated diseases including atherosclerosis, coronary heart disease, cerebrovascular disease, impaired renal function, peripheral vascular disease, Arteriovenous thrombosis disease, hypertension, hyperlipidemia, diabetes, psychiosis, acute cardio-cerebrovascular events, acute cerebrovascular events, cerebral infarction, cerebral hemorrhage, transient cerebral ischemic attack, transient cerebral ischemia, cerebral apoplexy, stroke, acute cardiovascular events, coronary heart disease, pectoris angina, acute myocardial infarction, cardiac shock, sudden death, and cardiogenic sudden death; said kit including no less than one kind of oligonucleotide fragment of claim 8 or claim 9, and suitable assay buffer system and color system.

19. The kit of claim 18, wherein said MTHFR gene polymorphisms also include the SNPs selected form A1298C, G1793A, G215A, G482A, and A1317G and any other polymorphisms that are in linkage disequilibrium with the above SNPs.

20. The kit of claim 18, wherein
(1) the TT genotype of MTHFR gene C677T polymorphism predicts an increased level of Hcy and an increased risk for hyperHcymia (high Hcy level);
(2) the CC genotype of MTHFR gene C677T polymorphism predicts a decreased level of Hcy and a decreased risk for hyperHcymia (high Hcy level);
(3) the TT genotype of MTHFR gene C677T polymorphism predicts an increased risk of developing Hcy-associated diseases and poorer prognosis of theses diseases;
(4) the CC and/or CT genotypes of MTHFR gene C677T polymorphism predicts an decreased risk of developing Hcy-associated diseases and better prognosis of theses diseases.

21. The kit of claim 20, wherein said subject refers to the subject with hypertension or with hyperhomocystinemia.

22. The kit of claim 21, wherein said subject refers to the subject with hypertension and with hyperhomocystinemia.

23. The kit of claim 18, wherein said biological specimen includes blood sample, body fluid sample, tissue sample, organ sample, and cultured cells, of which blood sample is the preferred biological specimen.

24. A usage of MTHFR gene polymorphisms in predicting treatment effect of medicine in a subject, said MTHFR gene polymorphisms including the C677T and/or A1298C polymorphism at least, said treatment effect includes increased homocysteine level and impaired liver function.

25. The usage of claim 24, wherein said medicine includes medications in the category of Angiotension Converting Enzyme Inhibihor (ACEI), which include but not limited to benazepril, captopril, enalapril, cilazapril, peridopril, delapril, quinapril, lisinopril, ramipril, imidapril, zofenopril, trandolapril and fosinopril, of which the preferred medication is enapril, benazepril, lisinopril or fosinopril.

26. The usage of claim 24, wherein said MTHFR gene polymorphisms also include the SNPs selected from G1793A, G215A, G482A, and A1317G and any other polymorphisms that are in linkage disequilibrium with the above SNPs.

27. The usage of claim 24, wherein
(1) subjects with the MTHFR 677TT homozygote genotype and/or 1298AA homozygote genotype tend to have greater increase in Hcy level induced by the treatment of ACEI medicine, subjects with the MTHFR 677CC homozygote genotype and/or 1298CC homozygote genotype tend to have smaller increase in Hcy level induced by the treatment of ACEI medicine;
(2) subjects with the MTHFR 677CC homozygote genotype tend to have greater impaired liver function induced by the treatment of ACEI medicine; subjects with the MTHFR 677TT homozygote genotype tend to have smaller impaired liver function induced by the treatment of ACEI medicine.

28. A usage of MTHFR gene polymorphisms in predicting treatment effects of medical compounds in a subject: said MTHFR gene polymorphisms including the C677T and/or A1298C polymorphism at least; said medical compounds including ACEI medicines and B vitamins; said treatment effects including (1) reducing homocysteine level; (2) reducing damage to liver function induced by ACEI medicine; (3) lowering blood pressure; and/or (4) protecting target organs.

29. The usage of claim 28, wherein said ACEI medicines include but not limited to benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinopril; said B vitamins include folic acid and its analogues, vitamin B6 and vitamin B12, of which the preferred B vitamin is folic acid.

30. The usage of claim 28, wherein said MTHFR gene polymorphisms also include the SNPs selected from G1793A, G215A, G482A, and A1317G and any other polymorphisms that are in linkage disequilibrium with the above SNPs.

31. The usage of claim 28, wherein
(1) the MTHFR 677TT homozygote genotype and/or 1298AA homozygote genotype predicts that said medical compound is likely to have a greater effect in lowering homocysteine level; the MTHFR 677CC homozygote genotype and/or 1298CC homozygote genotype predicts that said medical compound is likely to have a weaker effect in lowering homocysteine level;
(2) the MTHFR 677CC homozygote genotype predicts that said medical compound is likely to have a greater effect in reducing liver function damage induced by ACEI medicine; the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure; the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs; the MTHFR 677CC homozygote genotype and/or 1298CC homozygote genotype predicts that said medical compound is likely to have a weaker effect in protecting target organs.

32. The usage of claim 28, wherein said protecting target organs includes protection of renal function, prevention of re-stenosis after percutaneous transluminal coronary angioplasty (PTCA), prevention of hypertension and cardiovascular or cerebrovascular diseases associated complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, retinal artery sclerosis, retinal artery occulation, arteriosclerotic retinopathy, and arteriosclerotic retinitis.

33. A method of predicting the treatment effect of medicine in a subject using MTHFR gene polymorphisms, said MTHFR gene polymorphisms including the C677T and/or A1298C polymorphism at least, said medicine including ACEI medicine, said treatment effect including increased homocysteine level and damage to liver function, said method comprising the following steps of: (a) determining the genotypes of MTHFR gene polymorphisms by the oligonucleotide fragment of claim 8 or claim 9; (b) predicting the treatment effects of said ACEI medicines using the genotypes of MTHFR gene polymorphisms.

34. The method of claim 33, wherein said ACEI medicine include benazepril, captopril, enalapril, cilazapril, peridopril, delapril, quinapril, lisinopril, ramipril, imidapril, zofenopril, trandolapril and fosinopril, of which the preferred ACEI medicine is Enapril, benazepril, lisinopril or fosinopril.

35. The method of claim 33, wherein said MTHFR gene polymorphisms also include the SNPs selected form G1793A, G215A, G482A, and A1317G and any other polymorphisms that are in linkage disequilibrium with the above SNPs.

36. The method of claim 33, wherein
(1) subjects with the MTHFR 677TT homozygote genotype and/or 1298AA homozygote genotype tend to have greater increase in Hcy level induced by the treatment of ACEI medicine, subjects with the MTHFR 677CC homozygote genotype and/or 1298CC homozygote genotype tend to have smaller increase in Hcy level induced by the treatment of ACEI medicine;
(2) subjects with the MTHFR 677CC homozygote genotype tend to have greater impaired liver function induced by the treatment of ACEI medicine; subjects with the MTHFR 677TT homozygote genotype tend to have smaller impaired liver function induced by the treatment of ACEI medicine.

37. The method of claim 33, wherein said method may be derived from all kinds of nucleic acid analytical techniques as follows: polymerase chain reaction(PCR), polymerase chain reaction-restriction fragment length polymorphism(PCR-RFLP), PCR-allele specificity oligonucleotide probe (PCR-ASO), PCR- sequence specificity oligonucleotide (PCR-SSO), sequencing, PCR-sequence specificity primer (PCR-SSP), PCR-fluorometric method, PCR-finger-printing method, oligonucleotide ligation analysis, fluorescence energy resonance transfer detection, biochip, nucleic acid-chip, DNA-chip, mass spectrum, gene-scan, single strand conformation polymorphism (SSCP), denaturing gel gradient electrophoresis, enzyme or chemistry mismatch cutting method and Taqman method.

38. The method of claim 33, wherein said biological specimen includes blood sample, body fluid sample, tissue sample, organ sample and cultured cells, of which the preferred biological specimen is blood sample.

39. A method of predicting treatment effect of the medical compounds in a subject using MTHFR gene polymorphisms, said genotypes of MTHFR gene polymorphism including the C677T and/or A1298C polymorphism at least, said medical compounds including ACEI medicine and B vitamin, said treatment effect including (1) reducing the homocysteine level; (2) reducing liver damage induced by the ACEI medicine; (3) lowering blood pressure; and/or (4) protecting target organs, said method comprising the following steps of: (a) determining the genotypes of MTHFR gene polymorphisms by the oligonucleotide fragment of claim 8 or claim 9; (b) predicting the treatment effect of said medical compounds by the genotypes of MTHFR gene polymorphisms.

40. The method of claim 39, wherein said medical compounds include ACEI medicines and B vitamins, wherein said ACEI medicines include benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinoprill, wherein said B vitamins include folic acid and its analogues, vitamin B6, and vitamin B12, of which the preferred B vitamin is folic acid.

41. The method of claim 39, wherein said MTHFR gene polymorphisms also include the SNPs of G1793A, G215A, G482A, and A1317G and any other polymorphisms that are in linkage disequilibrium with the above SNPs.

42. The method of claim 39, wherein
(1) the MTHFR 677TT homozygote genotype and/or 1298AA homozygote genotype predicts that said medical compound is likely to have a greater effect in lowering homocysteine level; the MTHFR 677CC homozygote genotype and/or 1298CC homozygote genotype predicts that said medical compound is likely to have a weaker effect in lowering homocysteine level;
(2) the MTHFR 677CC homozygote genotype predicts that said medical compound is likely to have a greater effect in reducing liver function damage induced by ACEI medicine; the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure; the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs; the MTHFR 677CC homozygote genotype and/or 1298CC homozygote genotype predicts that said medical compound is likely to have a weaker effect in protecting target organs.

43. The usage of claim 39, wherein said protecting target organ includes protection of renal function, prevention of re-stenosis after percutaneous transluminal coronary angioplasty (PTCA), and prevention of hypertension and cardiovascular or cerebrovascular diseases associated complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, retinal artery sclerosis, retinal artery occulation, arteriosclerotic retinopathy, and arteriosclerotic retinitis.

44. The usage of claim 39, wherein said method may be derived from all kinds of nucleic acid analytical techniques as follows: polymerase chain reaction(PCR), polymerase chain reaction-restriction fragment length polymorphism(PCR-RFLP), PCR-allele specificity oligonucleotide probe (PCR-ASO), PCR-sequence specificity oligonucleotide (PCR-SSO), sequencing, PCR-sequence specificity primer (PCR-SSP), PCR-fluorometric method, PCR-finger-printing method, oligonucleotide ligation analysis, fluorescence energy resonance transfer detection, biochip, nucleic acid-chip, DNA-chip, mass spectrum, gene-scan, single strand conformation polymorphism (SSCP), denaturing gel gradient electrophoresis, enzyme or chemistry mismatch cutting method and Taqman method.

45. The usage of claim 39, wherein said biological specimen includes blood sample, body fluid sample, tissue sample, organ sample and cultured cells, of which the preferred specimen is blood sample.

46. A kit of predicting the treatment effects of medicine in a subject using MTHFR gene polymorphisms, said MTHFR gene polymorphisms include at least C677T and/or A1298C SNP, said medicine including ACEI medicines, said treatment effects including increased homocysteine level and damage to liver function induced by the ACEI medicine, said kit including no less than one kind of oligonucleotide fragment of the claim 8 or claim 9, and suitable assay buffer system and color system.

47. The kit of claim 46, wherein said ACEI medicines include benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinopril.

48. The kit of claim 46, wherein said MTHFR gene polymorphisms also include the SNPs selected from G1793A, G215A, G482A, and A1317G and any other polymorphisms that are in linkage disequilibrium with the above SNPs.

49. The kit of claim 46, wherein
(1) subjects with the MTHFR 677TT homozygote genotype and/or 1298AA homozygote genotype tend to have greater increase in Hcy level induced by the treatment of ACEI medicine, subjects with the MTHFR 677CC homozygote genotype and/or 1298CC homozygote genotype tend to have smaller increase in Hcy level induced by the treatment of ACEI medicine;
(2) subjects with the MTHFR 677CC homozygote genotype tend to have greater impaired liver function induced by the treatment of ACEI medicine; subjects with the MTHFR 677TT homozygote genotype tend to have smaller impaired liver function induced by the treatment of ACEI medicine.

50. A kit of predicting the treatment effect of medical compounds in a subject using MTHFR gene polymorphisms, said medical compounds including ACEI medicine and B vitamin, said treatment effect including (1) reducing the homocysteine level; (2) reducing liver damage induced by ACEI medicine; (3) lowering blood pressure; and/or (4) protecting target organ, said MTHFR gene polymorphisms including the C677T and/or A1298C polymorphism at least, said kit includes no less than one oligonucleotide fragment of the claim 8 or claim 9, and suitable assay buffer system and color system.

51. The kit of claim 50, wherein said medical compounds include ACEI medicines and B vitamins, wherein said ACEI medicines include benazepril, captopril, enalapril, cilazapril, peridopril, delapril, quinapril, lisinopril, ramipril, imidapril, zofenopril, trandolapril and fosinopril, of which the preferred ACEI medicine is enapril, benazepril, lisinopril or fosinopril, wherein said B vitamin includes folic acid and its analogues, vitamin B6 and vitamin B 12, of which the preferred B vitamin is folic acid.

52. The kit of claim 50, wherein said MTHFR gene polymorphisms also include the SNPs of G1793A, G215A, G482A, and A1317G and any other polymorphisms that are in linkage disequilibrium with the above SNPs.

53. The kit of claim 50, wherein
(1) the MTHFR 677TT homozygote genotype and/or 1298AA homozygote genotype predicts that said medical compound is likely to have a greater effect in lowering homocysteine level; the MTHFR 677CC homozygote genotype and/or 1298CC homozygote genotype predicts that said medical compound is likely to have a weaker effect in lowering homocysteine level;
(2) the MTHFR 677CC homozygote genotype predicts that said medical compound is likely to have a greater effect in reducing liver function damage induced by ACEI medicine; the MTHFR 677TT homozygote genotype predicts that said medical compound is likely to have a weaker effect in reducing liver function damage induced by ACEI medicine;
(3) the MTHFR 677TT homozygote genotype and/or 1298AA homozygote genotype predicts that said medical compound is likely to have a greater effect in lowering blood pressure; the MTHFR 677CC homozygote genotype and/or 1298CC homozygote genotype predicts that said medical compound is likely to have a weaker effect in lowering blood pressure;
(4) the MTHFR 677TT homozygote genotype and/or 1298AA homozygote genotype predicts that said medical compound is likely to have a greater effect in protecting target organs; the MTHFR 677CC homozygote genotype and/or 1298CC homozygote genotype predicts that said medical compound is likely to have a weaker effect in protecting target organs.

54. The kit of claim 50, wherein said protecting target organ includes protection of renal function, prevention of re-stenosis after percutaneous transluminal coronary angioplasty (PTCA), and prevention of hypertension and cardiovascular or cerebrovascular diseases associated complications such as artery sclerosis, coronary atherosclerosis, coronary heart disease, coronary atherosclerotic heart disease, angina pectoris, myocardial infarction, heart failure, peripheral vascular disease, cerebrovascular disease, cerebral hemorrhage, cerebral infarction, ischemic cerebrovascular disease, lacunar type of ischemic cerebrovascular disease, cerebral infarction, lacunar cerebral infarction, retinal artery sclerosis, retinal artery occulation, arteriosclerotic retinopathy, and arteriosclerotic retinitis.

55. A gene chip that includes the oligonucleotide fragment of the claim 8 or claim 9 for determining the genotypes of MTHFR gene polymorphisms.
